(19) United States
(12) Patent Application Publication
(10) Pub. No.: US 2012/0171770 A1
(43) Pub. Date: Jul. 5, 2012

(54) BIOENGINEERED SILK PROTEIN-BASED NUCLEIC ACID DELIVERY SYSTEMS

(75) Inventors: Keiji Numata, Medford, MA (US); David L. Kaplan, Concord, MA (US)

(73) Assignee: TRUSTEES OF TUFTS COLLEGE, Medford, MA (US)

(21) Appl. No.: 13/381,105

(22) PCT Filed: Jul. 9, 2010
(86) PCT No.: PCT/US10/41615

§ 371 Date: Mar. 20, 2012

Related U.S. Application Data
(60) Provisional application No. 61/224,618, filed on Jul. 10, 2009.

(51) Int. Cl. C12N 15/85; C12N 15/63; C07K 14/435; B82Y 5/00

(52) U.S. Cl. 435/455; 530/353; 435/320.1; 428/402; 977/773

(57) ABSTRACT

Nucleic acid transfer is achieved using a silk-based delivery system which releases nucleic acids from silk-based complexes. The silk-based complexes, which are composed, for example, of plasmid DNA (pDNA) and recombinant silk containing polycation and specific polypeptides sequences, can show high biocompatibility, high delivery efficiency, cell selectivity and controlled release of nucleic acid for nucleic acid transfection.

### CROSS REFERENCE TO RELATED APPLICATIONS

This application claims the benefit of priority of U.S. Provisional Application No. 61/224,618 filed Jul. 10, 2009, the content of which is incorporated herein by reference in its entirety.

### GOVERNMENT SUPPORT

This invention was made with funding under grant P41 EB002520, awarded by the National Institutes of Health (Tissue Engineering Resource Center). The U.S. government has certain rights in the invention.

### FIELD OF THE INVENTION

The present invention relates to molecular genetics, gene therapy, biopolymer nucleic acid delivery systems, and biomedicine. More specifically, the present embodiments provide for bioengineered silk proteins as a new family of highly tailored nucleic acid delivery systems.

### BACKGROUND

The concepts of gene therapy arose several decades ago as knowledge of the molecular mechanisms of genetic expression grew. With the arrival of recombinant DNA techniques, cloned genes became available and were used to demonstrate that foreign genes could indeed correct genetic defects and disease phenotypes in mammalian cells in vitro. Efficient retroviral vectors and other gene transfer methods have permitted convincing demonstrations of efficient phenotype correction in vitro and in vivo, now making gene therapy more broadly accepted as an approach to therapy. Recently, RNA interference or gene silencing has been a new way to degrade RNA of a particular sequence to treat various diseases. If a small-hairpin-RNA is designed to match the RNA copied from a faulty gene, then the abnormal protein product of that gene will not be produced.

Current nucleic acid delivery systems have drawbacks, however, including potentially severe allergic reactions caused by the introduction of viral vectors. Hence, there remains a need for alternative systems for delivering nucleic acids to a host cell or subject. More specifically, there is a need for a useful nonviral nucleic acid vector that is biocompatible, biodegradable, has low toxicity, high transfection/delivery efficiency, can be targetable to specific cell types and can modulate the controlled release of nucleic acids from the nucleic acid vector.

### SUMMARY

Silk proteins self-assemble into mechanically robust material structures that are also biodegradable and biocompatible, suggesting utility for nucleic acid delivery. Because silk proteins can also be tailored in terms of chemistry, molecular weight and other design features via genetic engineering, this system for nucleic acid delivery can be fine-tuned. The present invention is generally for nucleic acid delivery (e.g., plasmid DNA, small interfering RNA) to targeted cells.

In one embodiment, novel silk-based copolymers were bioengineered with poly(L-lysine) domains for nucleic acid delivery. The polymers self-assembled in solution and complexed with nucleic acids (e.g., DNA) through ionic interactions. In one embodiment, ionic complexes of these silk-polylysine-based copolymers complexed with DNA, successfully transfected genes to human cells. The material systems were characterized by agarose gel electrophoresis, atomic force microscopy, zeta-potentialmeter, confocal laser scanning microscopy and dynamic light scattering.

In another embodiment, novel silk-based matrices were bioengineered with one or more cell binding motifs (e.g., RGD domains) for nucleic acid delivery. The matrices complexed with nucleic acids and successfully transfected human cells with nucleic acid (e.g., pDNA).

In another embodiment, novel silk-based matrices were bioengineered with one or more domains of cell-penetrating and cell membrane-destabilizing peptides for nucleic acid delivery. The matrices complexed with nucleic acids and successfully transfected human cells with nucleic acids (e.g., pDNA), and present enhanced transfection efficiency, controlled enzymatic degradation rate, and controlled release of nucleic acids from the complexes.

The silk-based matrices can also be bioengineered with one or more other functional peptide domains, such as signal peptides of virus, tumor-homing peptides, metal binding domain, cell targeting peptides, drug binding peptides, functional domains to alter cell activities, and combinations thereof, to modulate delivery efficiency and selectivity and cell activities.

In a particular embodiment, silk-based biopolymer/nucleic acid complexes with 30-lysine residues prepared at a polymer/nucleotide molar ratio of 10:1 and with an average solution diameter of 380 nm, showed a high efficiency for cell transfection. The DNA complexes were also immobilized on silk films and demonstrated direct cell transfection from these surfaces.

In another particular embodiment, silk-based biopolymer/ nucleic acid complexes with 30-lysine residues and 11 RGD sequences, prepared at the ratio of number of amines to number of phosphates of DNA (referred to as N/P) of 2 and with an average solution diameter of 186 nm, showed a high cell transfection efficiency. The results demonstrate that bioengineered silk proteins are a new family of highly tailored nucleic acid delivery systems, and additional functional features can be added to the delivery systems to improve the delivery efficiency and selectivity.

In yet another embodiment, silk-based biopolymer/ nucleic acid complexes with polylysine and ppTG1 dimer sequences, prepared at the ratio of number of amines to number of phosphates of DNA (referred to as N/P) of 2, with a globular morphology and approximately 99 nm in diameter, showed a high cell efficiency. The dimeric sequence of ppTG1 significantly enhances transfection efficiency. The recombinant silks containing polylysine sequences and cell-penetrating and cell membrane-destabilizing peptides (CPPs) have useful transfection efficiency, comparable to the transfection reagent Lipofectamine 2000. These new bioengineered silk delivery systems can serve as a versatile and useful new platform polymer for non-viral nucleic acid delivery systems.

In addition, the secondary structure (e.g., transition to beta-sheet formation) of the silk sequence of the recombinant silk polymer/nucleic acid complexes is capable of controlling enzymatic degradation rates of the complexes, and hence can regulate release profiles of nucleic acids from the complexes.

The present invention is generally for nucleic acid (e.g., plasmid DNA delivery, small interfering RNA delivery) and drug delivery system to specific targetted cells. To further enhance the introduction efficiency and its specificity of the nucleic acid matrix to cells, specific peptide sequences targeting a certain disease, for example, cell binding motifs, cell penetrating peptides, signal peptides of virus, tumor-homing peptides, and metal binding domain for coating micro or nano magnetic particles to heat and kill disease cells, can be added into the recombinant silk.

The sizes of the nucleic acid complexes are also controlled by molecular weight of polylysine sequence or recombinant silk/nucleic acid ratio. To control induction time of gene transfection, the degradation rate of the gene complexes can also be controlled by the secondary structure of silk sequence of the recombinant silk. Recombinant silks modified to contain polylysine sequences form globular complexes with nucleic acids, for example, nano-particles, micelles, or micro capsules.

Further, the nucleic acid complexes immobilized on the surface of silk-based materials can be used as new nucleic acid delivery system. The versatility in both design and application of these new novel bioengineered silk protein-based delivery systems for nucleic acids or drugs provides utility in many delivery applications. For example, a silk matrix may be used as a bandage or insert, and also delivery a nucleic acid encoding a growth factor advantageous to tissue healing.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents a schematic presentation of a particular embodiment of the present invention: (A) plasmid DNA (pDNA) complex formation with silk-polylysine block copolymer; (B) preparation of a silk film containing pDNA complex; and (C) cell transfection using the silk film containing pDNA complex.

FIG. 2 shows the amino acid sequences of the silk6mer-lysine (recombinant spider silk protein) and poly-L-lysine sequences in an embodiment of the invention. Underline: representative monomeric spider silk unit.

FIG. 3 is an SDS-PAGE of the recombinant silk protein before (A) and after purification by Ni-NTA chromatography (B), where lane 1: Silk6mer, lane 2: Silk6mer-15lys, lane 3: Silk6mer-30lys, and lane 4: Silk6mer-45lys. lane M: molecular weight markers.

FIG. 4 shows AFM height images of Silk6mer-15lys proteins either (A) without pDNA or (B) with pDNA on silicon wafer substrates. (C): pDNA complexes with Silk6mer-30lys, (D): pDNA complexes with Silk6mer-45lys, and (E): pDNA complexes with Silk6mer. The pDNA complexes in this figure were prepared at P/N ratio of 10.

FIG. 5 is an agarose gel of pDNA and pDNA complexes with different molecular weights of lysine sequence (A) and different polymer/nucleotide (P/N) ratios (B). A1 and B1: pDNA (control), A2: Silk6mer and pDNA (P/N 10), A3: Silk6mer-lys15 and pDNA (P/N 10), 4: Silk6mer-lys30 and pDNA (P/N 10), A5: Silk6mer-lys45 and pDNAm (P/N 10), B2: Silk6mer-lys30 and pDNA (P/N 2.5), B3: Silk6mer-lys30 and pDNA (P/N 5), B4: Silk6mer-lys30 and pDNA (P/N 10),), and B5: Silk6mer-lys30 and pDNA (P/N 25), B6: Silk6mer-lys30 and pDNA (P/N 50).

FIG. 6 shows AFM height image of the surface of the silk film containing pDNA complexed with Silk6mer-30lys (A). (B) Line profile data of the white line in FIG. 6A.

FIG. 7 presents transfection results in loading pDNA complexes with different P/N ratio in HEK cells. Fluorescence microscopy images of cells incubated on the silk films containing pDNA complexes of Silk6mer30lys. (7A) P/N 2.5, (7B) P/N 5, (7C) P/N 10, (7D) P/N 25, and (7E) P/N 50. (7F) Plot of transfection efficiency loading pDNA complexes in HEK cells according to fluorescence images, and data are shown as means±standard deviation (n=4). *Significant difference between two groups at p<0.05.

FIG. 8 demonstrates transfection results in loading pDNA complexes with different polylysine sequences in HEK cells. Fluorescence microscopy images of cells incubated on the silk films containing pDNA complexes of Silk6mer (8A), Silk6mer-15lys (8B), Silk6mer-30lys (8C), and Silk6mer-45lys (8D). The green in the images represents successfully transfected cells. FIG. 8E) shows plot of transfection efficiency from the fluorescence images, and data are shown as means±standard deviation (n=4). *Significant difference between two groups at p<0.05.

FIG. 9 reflects cell viability after treatment of HEK cells with pDNA complexes (P/N=10) with different lysine sequences. Data are shown as means±standard deviation (n=8). *Significant difference between two groups at p<0.05.

FIG. 10 shows a schematic presentation of the strategy used for pDNA complex formation with silk-polylysine-RGD block copolymer, and cell transfection using the pDNA complex.

FIG. 11 presents amino acid sequences of the recombinant spider silk protein to contain poly-L-lysine and RGD sequences. Underline: representative monomeric spider silk unit.

FIG. 12 is a SDS-PAGE of the recombinant silk protein after purification by Ni-NTA chromatography. RS, RSR, SR, S2R, 11RS and molecular weight markers (M) are listed in each line.

FIG. 13 shows the dimensions and shapes of pDNA complexes of the recombinant silks. (13A) Average diameters of pDNA complexes of the recombinant silks determined by DLS as a function of polymer/pDNA (P/N) ratio. AFM height images of pDNA complexes with RS (13B) and RSR (13C) prepared at P/N ratio of 500 on mica.

FIG. 14 shows the electric charges of the pDNA complexes with the recombinant silks. Agaro se gel of pDNA and pDNA complexes of RSR with different P/N ratio (A) and pDNA complexes with different recombinant silks prepared at P/N of 500 (B). (C) Zeta potential of pDNA complexes of RSR as a function of polymer/pDNA (P/N) ratio.

FIG. 15 is the cell viability after treatment of HEK cells with pDNA complexes (P/N=500, N/P>10) with the different recombinant silks. Data are shown as means±standard deviation (n=8). *Significant difference between two groups at p<0.05.

FIG. 16 presents transfection results in loading DNA complexes of the recombinant silks with different P/N ratio in HEK cells (A). Data are shown as means±standard deviation (n=4). *Significant difference between two groups at p<0.05. (B, C) Fluorescence microscopy images of cells transfected through the pDNA complexes of 11RS prepared at P/N of 200. The green in the images represents successfully transfected cells.

FIG. 17 shows the size distribution of the recombinant silks and their complexes with pDNA determined by DLS.

FIG. 18 shows the amino acid sequences of the recombinant spider silk protein with poly-L-lysine and RGD sequences. The RGD sequences are bolded and the representative 6mer of the spider silk sequence is underlined.

FIG. 19 shows (19A) AFM height image of pDNA complexed with recombinant silk-polylysine-RGD (11RS) prepared at N/P ratio of 2 on mica; and (19B) line profile data of the white line in FIG. 19A.

FIG. 20 shows the electric charges of the pDNA complexes with the recombinant silk-polylysine-RGD. Agarose gel of pDNA and pDNA complexes of 11RS with different N/P ratios (20A) and pDNA complexes with different recombinant silks prepared at N/P of 2 (20B). (20C) Zeta potential of pDNA complexes of 11RS as a function of molar ratio of amines/phosphate of DNA (N/P).

FIG. 21A presents transfection results in loading pDNA complexes of the recombinant silk (11RS) at different N/P ratio in HeLa cells. FIG. 21B and C present transfection results in loading different recombinant silks (11RS, RS, RSR, SR and S2R) prepared at N/P 2 in Hela (21B) and HEK cells (21C), respectively. Silk6mer-30lys block copolymer (S) and LIPOFECTAMINE® 2000 transfection reagent were used as control samples. Data are shown as means±standard deviation (n=4). *Significant difference between two groups at p<0.05.

FIGS. 22A-22D show the intracellular distribution of pDNA complexes with the recombinant silk (11RS) in HeLa cells. FIG. 22A is an overlay of the three images (22B-22D); FIGS. 22B and 22C show the CLSM characterization of the cells incubated with DAPI (22B) and Cy5 label (22C); and FIG. 22D shows the phase contrast of the complexes in the cells. The CLSM observation was carried out using a 63× objective. pDNA was labeled with Cy5 (red), and the nuclei were stained with DAPI (blue). Each scale bar represents 10 μm.

FIG. 23A is a schematic of the recombinant silk protein sequence. FIG. 23B shows the amino acid sequences of the recombinant spider silk proteins with poly-L-lysine and ppTG1 sequences. The representative 6mer of spider silk sequence is underlined, and the ppTG1 sequence is bolded. FIG. 23C shows SDS-PAGE of the recombinant silk proteins after purification by Ni-NTA chromatography. In FIG. 23C, Molecular weight ladder (L), Silk-polylysine-ppTG1 monomer (M), and Silk-polylysine-ppTG1 dimer (D) are listed in each line.

FIG. 24 shows the FTIR-ATR spectra of Silk-polylysine-ppTG1 dimer before (blue line) and after the methanol treatment (gray line) for 24 h. An arrow indicates a peak at 1625 cm⁻¹ originated from beta-sheet structure.

FIG. 25 shows AFM height images of pDNA complexes with Silk-polylysine-ppTG1 dimer prepared at N/P ratio of 2 on mica.

FIG. 26 presents pDNA protection results from DNase I enzymes. Digestion of pDNA exposed to DNase I was measured for the pDNA complexes of Silk-polylysine-ppTG1 dimer with or without MeOH treatment. The lane number represents: (1) free pDNA only, (2) free pDNA and DNase, (3) free pDNA and alpha-chymotrypsin, (4) free pDNA and protease XIV, (5) pDNA complexes of Silk-polylysine-ppTG1 dimer and DNase, (6) pDNA complexes of Silk-polylysine-ppTG1 dimer and protease XIV after DNase treatment, (7) pDNA complexes of Silk-polylysine-ppTG1 dimer and alpha-chymotrypsin, (8) pDNA complexes of Silk-polylysine-ppTG1 dimer and protease XIV, (9) MeOH-treated pDNA complexes of Silk-polylysine-ppTG1 dimer and DNase, (10) MeOH-treated pDNA complexes of Silk-polylysine-ppTG1 dimer and protease XIV after DNase treatment, (11) MeOH-treated pDNA complexes of Silk-polylysine-ppTG1 dimer and alpha-chymotrypsin, and (12) MeOH-treated pDNA complexes of Silk-polylysine-ppTG1 dimer and protease XIV.

FIGS. 27A-27D present the transfection results in loading pDNA complexes of Silk-polylysine-ppTG1 in HEK and MDA-MB-435 cells. FIG. 27A shows the transfection results of Silk-polylysine-ppTG1 dimer at different N/P ratios in HEK cells. FIG. 27B shows the transfection results of Silk-polylysine-ppTG1 monomer and dimer prepared at N/P 2 in HEK and MDA-MB-435 cells. Lipofectamine 2000 were used as positive control samples. Data are shown as means±standard deviation (n=4). *Significant difference between two groups at p<0.05. FIGS. 27C and 27D show the cell morphology after transfection with a DNA encoding GFP reporter gene complexed with Silk-polylysine-ppTG1 dimer prepared at N/P 2 to HEK cells (27C) and MDA-MB-435 cells (27D), respectively.

FIG. 28 presents the time course of transfection with the pDNA complexes of Silk-polylysine-ppTG1 dimer prepared at N/P 2 before (square) and after (triangle) MeOH treatment for 24 h. *Significant difference between two groups at p<0.05.

### DETAILED DESCRIPTION

This invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Gene therapy requires efficient and safe carriers to transfer nucleic acid into target cells. There are currently no Food and Drug Administration (FDA)-approved gene therapies, even though over 1,400 gene therapy clinical trials have been conducted since 1989. Gene The. Clin. Trials Worldwide, J. Gene Med. (2009). Viral vectors, including adenovirus and adeno-associated virus, have been used in gene delivery due to their relatively high efficiency of transfection and potential long term effects through integration into the host genome. Lundstrom, 21 Trends Biotech. 117-22 (2003). However, safety concerns remain about immune responses by the introduction of viruses as carriers. Moreover, using retroviruses in gene therapy can lead to complications such as leukemia, because genes of the virus can be inserted into any arbitrary position in the genome of the host. Edelstein et al., 6 J. Gene. Med. 597-602 (2004).

Silk proteins have been used successfully in the biomedical field as sutures for decades, and also explored as biomaterials for cell culture and tissue engineering, achieving FDA approval for such expanded utility because of excellent mechanical properties, versatility in processing and biocompatibility. Kaplan et al., ACS Symp. Ser. 544 (1994); Altman et al., 24 Biomats. 401-16 (2003); Wang et al., 27 Biomats. 6064-82 (2006). Furthermore, the degradation products of silk proteins with beta-sheet structures, when exposed to alpha-chymotrypsin, have recently been reported and show no cytotoxicity to in vitro neuron cells. Hollander, 43 Med. Hypotheses 155-56 (1994); Wen et al., 65 Ann. Allergy 375-78 (1990); Kurosaki et al., 66 Nippon Ika Daigaku Zasshi 41-44 (1999); Rossitch et al., 3 Childs Nerv. Sys. 375-78 (1987); Dewair et al., 76 J. Allergy Clin. Immunol. 537-42 (1985); Zaoming et al.,6 J. Invest. Aller. Clin. Immunol. 6 237-41 (1995); Numata et al., 31 Biomats. 2926-33 (2010).

Silk proteins are commonly produced by insects and spiders, form fibrous materials in nature, and have been used as medical sutures because of their excellent mechanical properties and biocompatibility. Kaplan et al., ACS Symp. Ser. 544 (1994). Beyond traditional uses, silk fibroin has also been explored as a biomaterial for cell culture and tissue engineering and achieved FDA approval for such expanded utility. Altman et al., 24 Biomats. 401-16 (2003); Wang et al., 27 Biomats. 6064-82 (2006).

Silk proteins modified by genetic engineering have are capable of displaying new features alongside the native properties. Wong et al., 54 Adv. Drug Deliv. Rev. 1131-43 (2002); Cappello et al., 3 Biotechnol. Prog. 198-202 (1990); Megeed et al., 54 Adv. Drug Deliv. Rev. 1075-91 (2002). For example, homoblock protein polymers consisting of silk-like crystalline blocks and elastin-like flexible blocks were generated to demonstrate the potential of combining the unique mechanical properties of silk and elastin proteins. Cappello et al., 1990; Megreed et al., 2002. Applications for release of adenoviral vectors containing plasmid DNA (pDNA) from these silk-elastin-like polymer hydrogels were also reported. Megeed et al., 94 J. Control Release 433-45 (2004). Modified spider silks bioengineered to include RGD cell-binding domains to enhance cell adhesion have also been reported. Bini et al., 7 Biomacromolecules 3139-45 (2006). Furthermore, biomaterial scaffolds prepared from this modified silk protein displayed enhanced ability to differentiate human bone marrow derived mesenchymal stem cells with regard to osteogenic outcomes. Id.

Many other examples of bioengineered silks can be described, from inclusion of molecular triggers to control of self-assembly (Szela et al., 1 Biomacromol. 534-42 (2000); Winkler et al., 39 Biochem. 12739-46 (2000)), chimeric silk proteins for controlled mineralization (Wong et al., 103 P.N.A.S. 9428-33 (2006); Huang et al., 28 Biomaterials 2358-67 (2007)), and recent all silk block copolymer designs. Rabotyagova et al., 10 Biomacromol. 229-36 (2009).

The secondary structure of silk fibroin generally determines the solubility and biodegradability of the material. Huemmerich et al., 43 Biochem. 13604-12 (2004). α-helix and random coil structures enhance solubility of silk fibroin in aqueous solutions, whereas β-sheet structures prevent silk protein from dissolving in aqueous solutions. Id. In addition, the degradation rate of silk fibroin increases with decreased β-sheet content. Li et al., 24 Biomats. 357-65 (2003). Beta sheet crystalline structure of silk protein can be induced by methods known to one skilled in the art, such as methanol treatment, water annealing treatment, lowering pH, applying electric field, applying shearing force, and the like.

RGD sequence, arginine-glycine-aspartic acid, is known to selectively recognize and bind αvβ3 and αvβ5 integrins that are expressed on cell surfaces of certain cell types such as endothelial cells, osteoclast, macrophage, platelets, and melanomas. Oba, Bioconjugate Chem. (2006); Kim, J. Controlled Release (2005); Connelly, Biomats. (2007). The integrins are considered to be a class of transmembrane glycoproteins that interact with the extracellular matrix, and are exploited for cell-binding and entry by receptor-mediated endocytosis, which is a representative pathway for gene delivery systems. Renigunta et al., 17 Bioconj. Chem. 327-34 (2006). RGD sequences are therefore a useful candidate as a ligand for gene vectors used for nucleic acid (e.g., plasmid DNA or siRNA) deliveries.

Cationic polymers and poly(amino acid)s can interact with nucleic acids through electrostatic interactions to assemble into polyelectrolyte complexes, which have been proposed as an alternative to recombinant viruses for the delivery of pDNA into cells. Zauner et al., 30 Adv. Drug Deliv. Rev. 97-113 (1998); Ogris et al., 6 Gene Ther. 595-605 (1999); Oupicky et al., 10 Bioconjug. Chem. 764-72 (1999); Breitenkamp et al., 9 Biomacromolecules 2495-2500 (2008); Fischer et al., 16 Pharm. Res. 1273-79 (1999); Ahn et al., 80 J. Controlled Release 273-82 (2002); Lavertu et al., 27 Biomats. 4815-24 (2006). Nucleic acid delivery is an attractive approach for a variety of disease states because, for example, an introduced gene may generate bioactive proteins in the modified host cells. Poly(L-lysine), which is degraded by cells, has been used as a cationic polymer to form delivery vehicles (vectors) for small drugs. Zauner et al., 1998. The interaction of polylysine with DNA has been characterized in detail by agarose gel electrophoresis (charge and size), electron microscopy (shape and size), atomic force microscopy (AFM) (shape and size), and dynamic light scattering (DLS) (size and size distribution). Zauner et al., 1998. Positively charged complexes can potentially induce cytotoxicity and form aggregates in biological media containing plasma proteins (Ogris et al., 1999; Oupicky et al., 1999), but the present DNA complexes of silk-based poly(L-lysine) copolymers containing less than 30 lysines showed no cytotoxicity to human embryonic kidney (HEK) cells.

A useful nonviral nucleic acid vector is biocompatible, biodegradable, has low toxicity and can be targetable to specific cell types. These are challenging design goals to meet with synthetic polymers. Different cationic block copolymers as gene vectors have been studied in recent years, including cationic liposomes, polylysine copolymers, polyethyleneimine (PEI) copolymers, and polysaccharides. Zauner et al., 1998; Breitenkamp et al., 2008; Fischer et al., 1999; Ahn et al., 2002); Lavertu et al., 2006. Natural biopolymers, in particular, are increasingly attractive as nonviral vectors because of their non toxicity and biocompatibility. Silk-based polymers, which can have added functions through bioengineering, offer an efficient biomaterial platform for tailoring chemistry, molecular weight, and targeting based on specific designs, thus can be a useful nonviral nucleic acid carriers.

The present invention provides for novel silk-based, non-viral nucleic acid vectors which are biocompatible, biodegradable, and utilize non-toxic cationic polymers. Silk-based polymers are useful candidates for nonviral nucleic acid vector, because functions can be added through recombinant techniques, offering a highly efficient approach to tailor chemistry, molecular weight and targeting based on system design. For another example, cell binding motifs (RGD), cell penetrating peptides (Elmquist et al., 269 Exp. Cell Res. 237-44 (2001); Rittner et al., 5 Mol. Ther. 104-14 (2002); Jarver et al., 35 Biochem. Soc'y Trans. 770-74 (2007)); signal peptides of virus (Makela et al., 80 J. Virol. 6603-11 (2006)); tumor-homing peptides (Laakkonen et al., 8 Nat. Med. 751-55 (2002); Porkka et al., 99 P.N.A.S. 7444-49 (2002); Christian et al., 163 J. Cell Biol. 871-78 (2003); Laakkonen et al., 101 P.N.A.S. 9381-86 (2004); Pilch et al.,103 P.N.A.S. 2800-04 (2006)); and metal binding domain for coating micro or nano magnetic particles to heat and kill disease cells (Obradors et al., 258 Eur. J. Biochem. 207-13 (1998); Park et al., 128 J. Am. Chem. Soc'y 7938-46 (2006)), can be added into the recombinant silk to enhance the cell transfection efficiency and its cell selectivity.

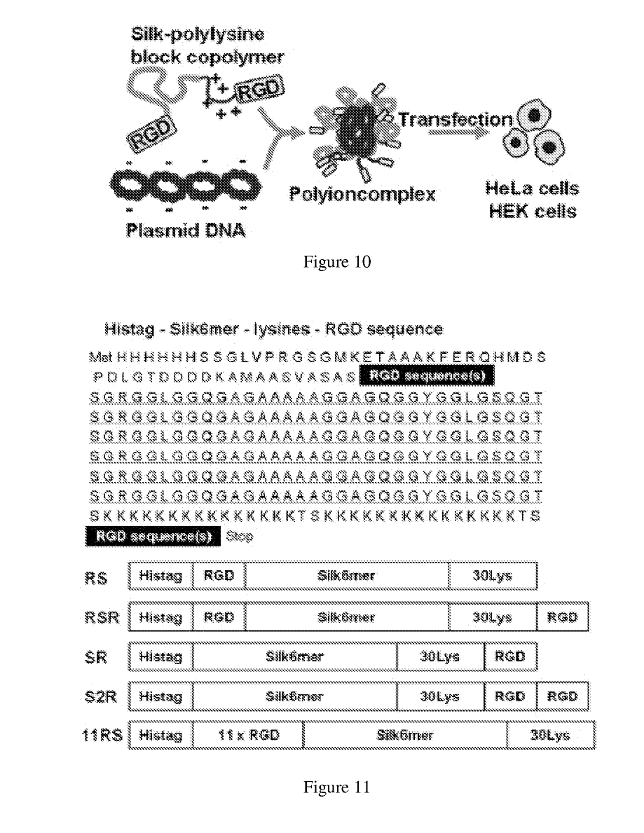

An embodiment of the present invention enhances transfection efficiency of the silk-based nucleic acid vectors, which are biocompatible, biodegradable, and utilize non-toxic cationic polymers, by an addition of one or more cell-binding motifs, e.g., RGD, into the recombinant silk sequence. This also provides for the influences of positions of RGD sequences, such as C-terminus and N-terminus, on the transfection efficiency to cells, which is valuable information to consider when constructing novel protein-based nucleic acid vectors. Complexes of a silk-based copolymer with plasmid DNA were prepared for in vitro nucleic acid delivery to HeLa and HEK cells (FIG. 10), and characterized by agarose gel electrophoresis, zeta potentialmeter, Atomic Force Microscopy (AFM), and Dynamic Light Scattering (DLS).

One embodiment of the invention provides a less-cytotoxic and highly efficient nucleic acid carrier with enhanced transfection efficiency, by addition of one or more CPPs, e.g., ppTG1 peptide, into the recombinant silk sequence of the silk-based nucleic acid vector. ppTG1 peptide, a lysine-rich cell membrane-destabilizing peptide to bind pDNA, destabilizes the cell membrane and promotes nucleic acid transfer.

In a particular embodiment, genetically engineered silk proteins containing polylysine and the monomeric and dimeric ppTG1 sequences were synthesized in E. coli, followed by transfection experiments in human embryonic kidney cells, the level of which is comparable to the transfection regent Lipofectamine 2000. The assemblies of the nucleic acid complexed with the recombinant silk show a globular morphology with an average hydrodynamic diameter of 99 nm and almost no beta-sheet structure. Moreover, the silk-based nucleic acid complexes show excellent DNase resistance as well as efficient release of the nucleic acid by enzymes that degrade silk proteins. Additionally, comparison with beta-sheet induced silk-based nucleic acid complexes indicates the secondary structure of the silk sequence of the nucleic acid complexes controls the enzymatic degradation rate of the complexes, and hence can regulate the release profile of nucleic acids from the complexes. The bioengineered silk-based nucleic acid delivery vehicles containing cell membrane-destabilizing peptides therefore provide a less-toxic and controlled-release nucleic acid delivery system.

Recombinant silks modified to contain polylysine sequences form globular complexes with nucleic acids, for example, nano-particles, micelles, or micro capsules. The nucleic acid matrix can show effective and selective transfection of nucleic acids to cells. Silk-based biomaterials containing the nucleic acid complexes immobilized on their surface can also be used for direct transfection of nucleic acid to cells. The sizes of the charge nucleic acid complexes of silk-based block copolymers can be controllable based on the polymer/nucleic acid ratio or the molecular weight of the polylysine domain bioengineered into the designs. To control induction time of nucleic acid transfection, the degradation rate of the nucleic acid complexes can also be controlled by the secondary structure of silk sequence of the recombinant silk.

Any nucleic acid that provides for or mediates the expression of a protein or modulates cellular function is within the scope of the present invention. Hence, nucleic acid may refer to RNA, DNA, siRNA, RNA/DNA chimera, natural and artificial nucleotides or sequences, or combinations of these, and the like, without limitation. For example, the nucleic acid to be complexed with the recombinant silk include, but are not limited to, dsRNA (double-stranded RNA) siRNA (small interfering RNA), shRNA (short hairpin RNA), saRNA (small activating RNA), mRNA (Messenger RNA), miRNA (micro RNA), pre-miRNA, ribozyme, antisense RNA, DNA, cDNA, DNA or RNA vectors/plasmids, etc.

A plurality of amino acids that comprise positively charge side chains (R groups) can be used to modify silk protein to form recombinant silk sequence (silk-based copolymer). In one embodiment, the recombinant silk sequence are modified by one or more domains of lysine or arginine rich peptides, e.g., polylysine.

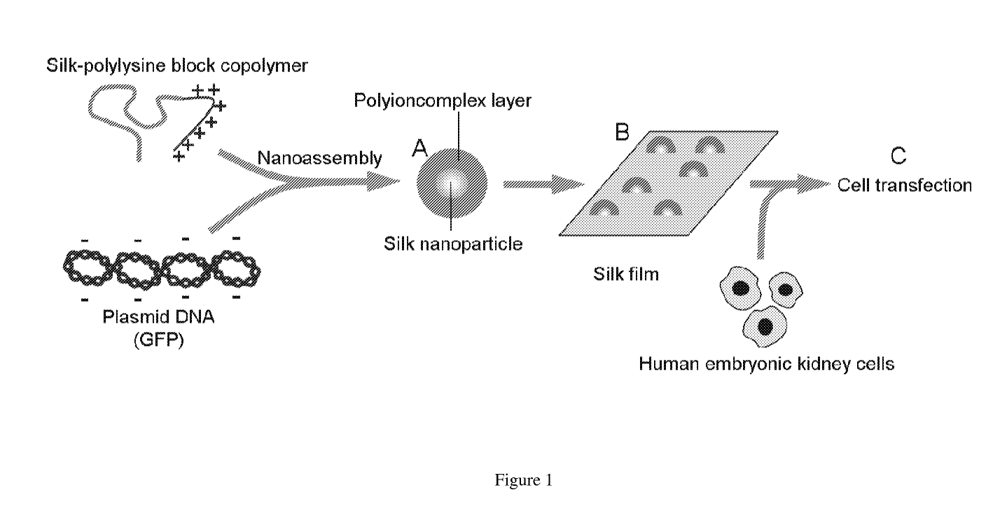

In a particular embodiment, a novel silk-based block copolymer combining spider silk and poly(L-lysine) was designed, generated, and characterized. Complexes of these silk-based block copolymers with plasmid DNA were prepared for in vitro nucleic acid delivery to HEK cells (FIG. 1), and characterized by agarose gel electrophoresis, Atomic Force Microscopy (AFM), and Dynamic Light Scattering (DLS). Silk films containing the DNA complexes were also prepared and cell transfection experiments were carried out on these films. When considering the novel polymer properties of silks, in terms of self-assembly, robust mechanical properties and controllable rates of degradation, in combination with tailored ionic complexation with nucleic acids and options for cell targeting reported here, a new family of vehicles for studies of nucleic acid delivery is described. Further, the nucleic acid complexes immobilized on the surface of silk-based materials can be used as new nucleic acid delivery system.

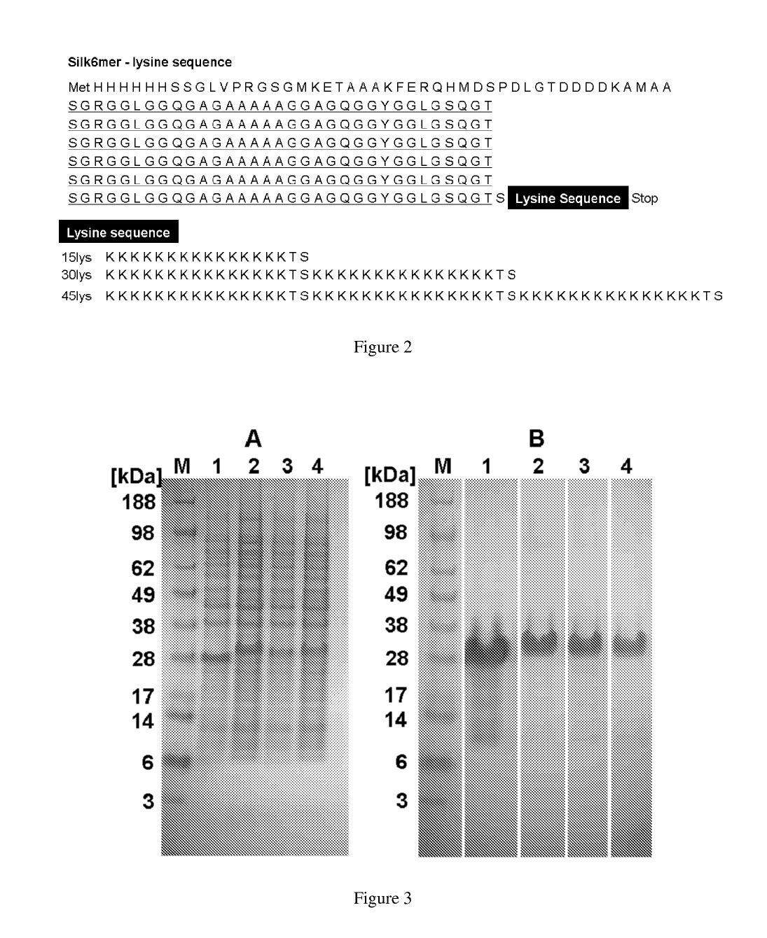

For expression and purification of silk protein, the amino acid sequences of the four spider silk variants generated, with and without polylysine, are shown in FIG. 2. Yields of the recombinant silk proteins were approximately 10 mg/L after purification and dialysis. The proteins before and after purification by Ni-NTA chromatography were analyzed by SDS-PAGE and stained with Colloidal blue to evaluate purity (FIG. 3). The Silk6mer control showed a band corresponding to a molecular weight of approximately 27 kDa (FIGS. 3A and 3B, lane 1). The recombinant silk proteins containing the lysine sequences, Silk6mer-15lys, Silk6mer-30lys, and Silk6mer-45lys, also showed molecular weights of around 30 kDa (FIG. 3A and B, lanes 2, 3, and 4), which was in accord with the theoretical molecular weights of 23, 25, and 27 kDa, respectively. The results of protein identification by LC/MS/MS using the gel bands confirmed that the bioengineered proteins were the expected recombinant silk proteins. The recombinant proteins were partially soluble in water and also soluble in HFIP (10 mg/mL) at room temperature.

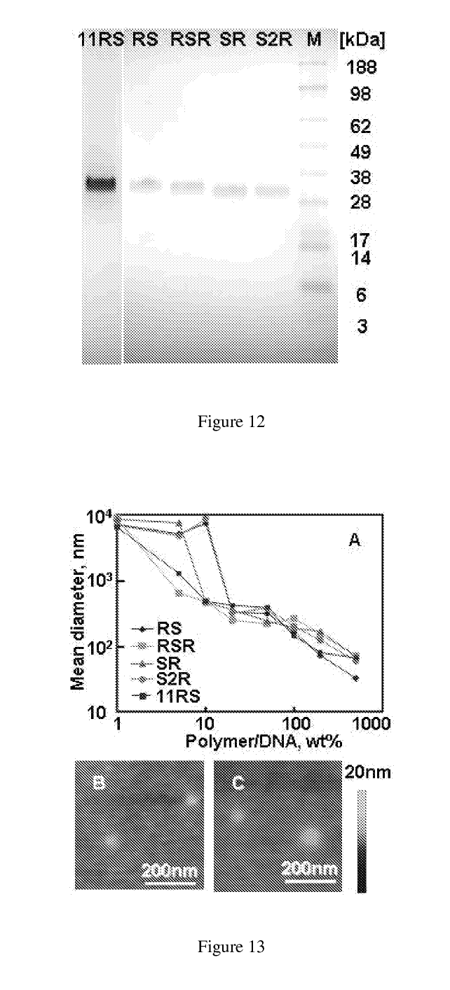

Additionally, the amino acid sequences of the five spider silk variants generated with polylysine and RGD cell-binding motifs (RS, RSR, SR, S2R, and 11RS) were generated, as shown in FIG. 11. Yields of the RGD-recombinant silk proteins were approximately 10 mg/L after purification and dialysis. The proteins before and after purification by Ni-NTA chromatography were analyzed by SDS-PAGE and stained with Colloidal blue to evaluate purity. RS, RSR, SR, S2R, and 11RS showed a band corresponding to a molecular weight of approximately 33, 32, 30, 30, and 35 kDa (FIG. 12), which was not in perfectly accord with the theoretical molecular weights (monoisotopic mass) of 26068.1, 26584.4, 25565.9, 26082.1, 31669.86 Da, respectively. The results of MALDI-TOF, however, showed 26068.1, 26584.4, 25565.9, 26082.1, and 31669.9 Da, respectively, and confirmed that the bioengineered proteins were the expected recombinant RGD-silk proteins. The recombinant proteins showed the theoretical pI of 10.6 and were soluble in water (1.0 mg/mL) at room temperature.

DNA-Protein complex formation with DNA encoding GFP with the four types of recombinant silk proteins (Silk6mer, Silk6mer-15lys, Silk6mer-30lys, and Silk6mer-45lys) and the five types of recombinant RDG-silk proteins (RS, RSR, SR, S2R, and 11RS), was characterized by AFM, DLS, and agarose gel electrophoresis.

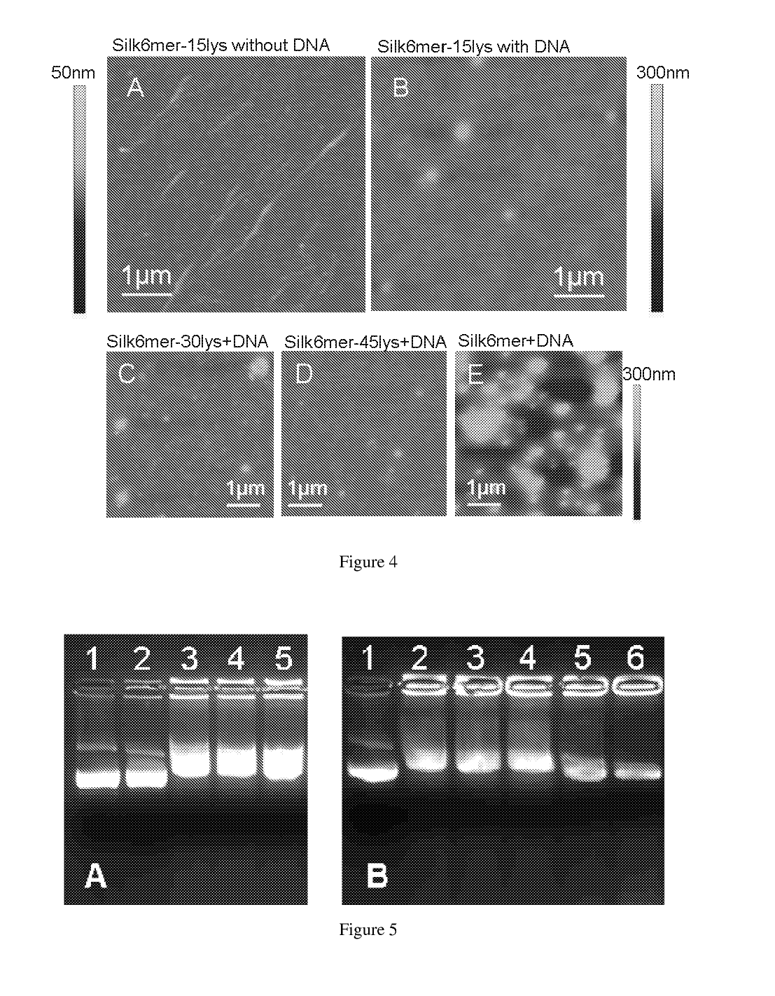

FIG. 4 shows a typical AFM height image of the DNA complexes with the recombinant silks (P/N 10) cast on a silicon wafer. Silk6mer-15lys molecules without pDNA were linear (FIG. 4A), whereas Silk6mer-15lys with DNA formed globular complexes (FIG. 4B). Further, globular complexes were also observed with the Silk6mer-30lys and Silk6mer-45lys (FIGS. 4C and 4D). The average diameter of the DNA complexes for Silk6mer-15lys, Silk6mer-30lys, and Silk6mer-45lys were 335±104 nm, 392±77 nm, and 436±91 nm, respectively (Table 1). On the other hand, Silk6mer molecules randomly aggregated with DNA (FIG. 4E), and the resulting features were not globular complexes but large aggregates with a diameter of 857±290 nm. Also, the statistical analysis of the dimensions of DNA complexes determined by AFM demonstrated significance differences between the complexes of Silk6mer and the other samples as shown in Table 1:

TABLE 1
Dimensions of the pDNA complexes of the recombinant silks
(P/N 10) determined by AFM

| Recombinant silk of complex | Width, nm |
|---|---|
| Silk6mer-15lys | 335 ± 104^{a} |
| Silk6mer-30lys | 392 ± 77^{a} |
| Silk6mer-45lys | 436 ± 91^{a} |
| Silk6mer | 857 ± 290^{b} |

Values are mean ± standard deviation, n = 30.
^{a,b}Statistically significant differences were seen between groups that are not sharing common superscripts at p < 0.05

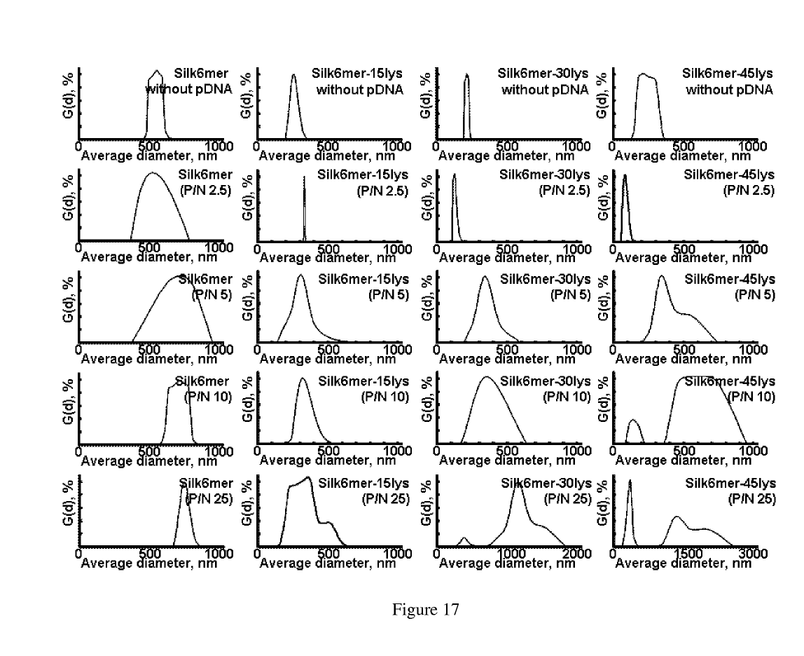

The hydrodynamic diameter of the recombinant silks and their pDNA complex were measured by DLS. (Table 2 and FIG. 17). The average diameters of Silk6mer without and with DNA were 570 nm and around 550-790 nm, respectively. The other three types of recombinant silks containing polylysine showed an average diameter of around 210-270 nm without DNA. The diameter of DNA complexes of the recombinant silk with polylysine sequences increased with increase in polylysine sequence or P/N ratio. In the case of Silk6mer-30lys (P/N 25) and Silk6mer-45lys (P/N 10 and 25), the diameters were bimodal, indicating both small and large complexes. The DNA complexes prepared at P/N 50 resulted in large precipitates and were not able to be characterized by DLS.

TABLE 2
Average diameters of the recombinant silks
and their complexes determined by DLS.

| Polymer | P/N | Diameter (nm) |
|---|---|---|
| Silk6mer | —^{a} | 570 |
| | 2.5 | 550 |
| | 5 | 790 |
| | 10 | 730 |
| | 25 | 750 |
| | 50 | 780 |
| Silk6mer-15lys | —^{a} | 270 |
| | 2.5 | 320 |
| | 5 | 280 |
| | 10 | 310 |
| | 25 | 380 |
| | 50 | —^{b} |
| Silk6mer-30lys | —^{a} | 210 |
| | 2.5 | 120 |
| | 5 | 330 |
| | 10 | 380 |
| | 25 | 400, 1150 |
| | 50 | —^{b} |
| Silk6mer-45lys | —^{a} | 210 |
| | 2.5 | 80 |
| | 5 | 370 |
| | 10 | 140, 590 |
| | 25 | 350, 1320 |
| | 50 | —^{b} |

^{a}The recombinant silk molecules without pDNA.
^{b}There were too much precipitation for analysis by DLS.

Similarly, the hydrodynamic diameters of DNA complex of the recombinant RGD-silks were measured by DLS (FIG. 4A) as shown in Table 3:

TABLE 3
Mean diameters distribution of DNA complex of recombinant silks determined by DLS

| P/N | 0 | 1 | 5 | 10 | 20 | 50 | 100 | 200 | 500 |
|---|---|---|---|---|---|---|---|---|---|
| RS | 6671 | 7079 | 5093 | 7317 | 328 | 316 | 164 | 71 | 32 |
| | (2.389) | (—) | (3.496) | (—) | (1.025) | (1.746) | (0.420) | (1.054) | (1.472) |
| RSR | 7572 | 8913 | 628 | 485 | 250 | 218 | 266 | 149 | 72 |
| | (—) | (6.05) | (1.327) | (2.021) | (1.22) | (1.103) | (0.271) | (0.871) | (1.079) |
| SR | 8273 | 8574 | 7469 | 461 | 351 | 238 | 189 | 165 | 68 |
| | (—) | (—) | (4.671) | (1.012) | (0.875) | (0.375) | (0.623) | (0.464) | (1.083) |
| S2R | 6460 | 6942 | 4735 | 8584 | 320 | 376 | 198 | 121 | 59 |
| | (3.862) | (—) | (5.801) | (3.325) | (0.666) | (0.511) | (0.807) | (0.556) | (0.726) |
| 10RS | 6998 | 6531 | 1266 | 480 | 417 | 382 | 144 | 79 | 66 |
| | (2.148) | (1.855) | (1.082) | (0.164) | (0.323) | (0.164 | (0.875) | (0.517) | (0.521) |

The average diameters of the complexes decreased with an increase in P/N ratio, resulting that the average diameters of RS, RSR, SR, S2R, and 11RS prepared at P/N of 500 were 32, 72, 68, 59 and 66 nm, respectively. The DNA complexes of RS and RSR prepared at P/N 500, which showed the smallest and largest diameters by DLS, were cast on mica and observed by AFM. RS and RSR with DNA formed globular complexes (FIGS. 13B and 13C). The average diameter of the DNA complexes for RS and RSR were 58±28 nm and 73±12 nm, respectively. The statistical analysis of the dimensions of DNA complexes determined by AFM demonstrated no significance differences between RS and RSR (p=0.12).

Agarose gel electrophoresis experiments were performed to investigate the interaction properties and electrolytic stabilities of the complexes of pDNA and recombinant silk polylysine. FIG. 5A shows the migration of free pDNA (lane 1) and the DNA complexes of the recombinant silks in 1% agarose gels (lanes 2-5). The migration of Silk6mer mixed with DNA demonstrated that free DNA was still present along with the Silk6mer molecules, whereas the recombinant silks containing polylysine sequences showed bands in the wells and migrated slower than free DNA, indicating that the DNA was partially bound on the recombinant silks; some release of pDNA may have occurred during electrophoresis. The mixtures of DNA and Silk6mer-30lys with various P/N ratios were analyzed by agarose gel electrophoresis (FIG. 5B). The P/N ratios ranging from 2.5 to 50 showed little variations in gel migration, indicating that the stability of the complexes were similar between these P/N ratios.

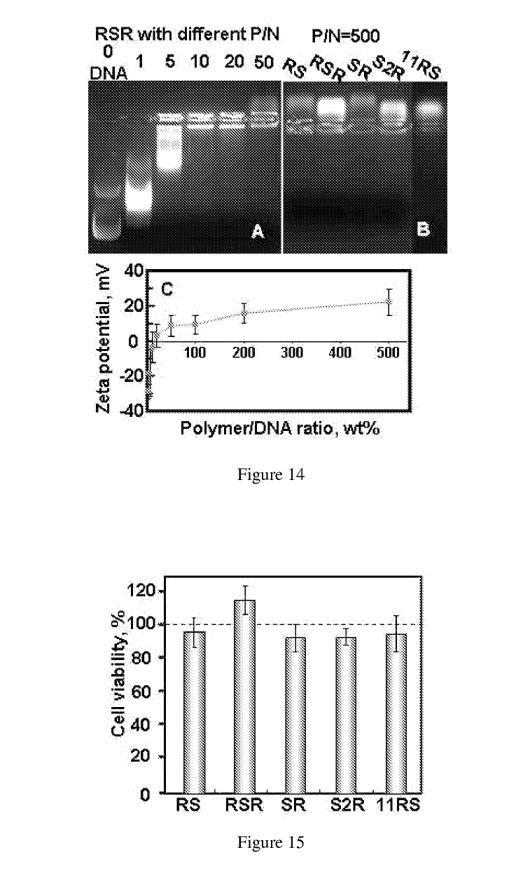

Agarose gel electrophoresis experiments were also performed to investigate the interaction properties and electrolytic stabilities of the complexes of DNA and recombinant RGD-silks. FIG. 14A shows the migration of free DNA and the DNA complexes of RSR with various P/N molar ratio ranging from 1 to 50 in 1% agarose gel. The DNA to form complexes with RSR at P/N ranged from 1 and 20 migrated to the same direction as free DNA or did not migrate from the well, whereas the complexes at P/N over 50 migrated to the opposite direction, indicating that these DNA complexes with RSR at P/N below 25 were negatively or neutrally charged, while the complexes at P/N over 50 were positively charged. The DNA complexes of four recombinant silks prepared at P/N 500 were also characterized by agarose gel electrophoresis, and the all five samples demonstrated positive charges. To measure values of the positive charge, zeta potential of the DNA complexes was measured. FIG. 14C shows the zeta potential of DNA complexes of RSR with varying P/N ratio. The zeta potential increased with P/N ratio, and became positive value at the P/N of 50. The zeta potential of P/N 50 and 500 was 8.58±5.47 and 22.2±4.03 mV.

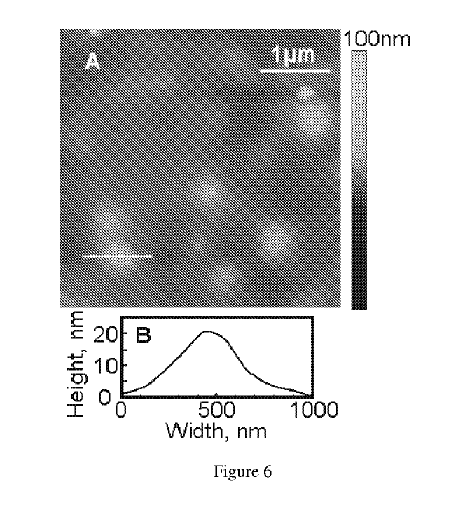

In a particular embodiment, the complexes of pDNA and recombinant polylysine silks were deposited as cast silk films. After washing the silk film with water to remove free pDNA, the surface of the silk films containing pDNA complexes was examined by AFM to evaluate the integrity of the complexes. FIG. 6 shows the AFM height image of the surface of the Silk6mer30-lys film containing pDNA complexes of Silk6mer-30lys. The particles were nearly identical in the size with the pDNA complex images acquired before casting on films (FIG. 4C), confirming the integrity of the particles after being cast on the films. It is also evident from FIG. 6 that the complexes were individually immobilized on the surface of silk film. As shown in FIG. 6B, the pDNA complexes were adsorbed on the surface, and the height of the complexes was approximately 20 nm.

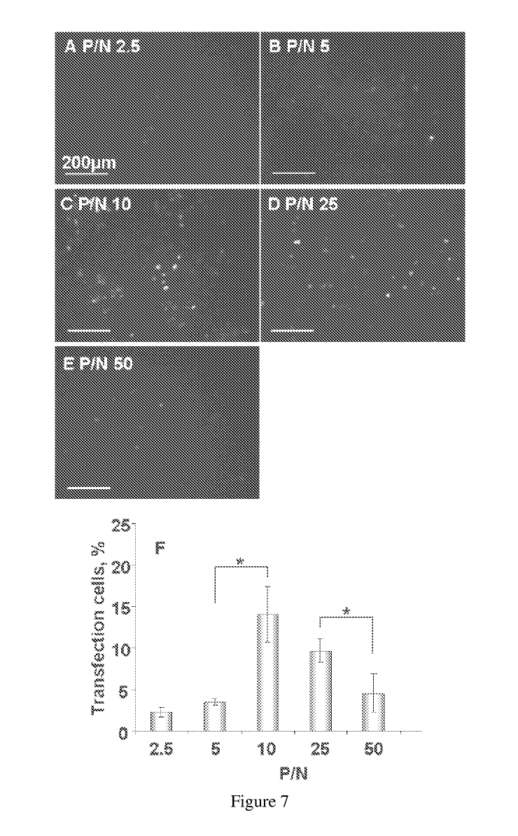

To evaluate the feasibility of the nucleic acid complexes with the cationic recombinant silks for nucleic acid delivery, in vitro transfection experiments were carried out with HEK cells. For a comparison of DNA transfection efficiency of various DNA complexes with different P/N ratio, HEK cells were transfected with GFP DNA as a reporter. FIG. 7 shows fluorescence microscopy images of cells incubated on the silk films containing pDNA complexes of Silk6mer-30lys prepared at P/N 2.5 (7A), P/N 5 (7B), P/N 10 (7C), P/N 25 (7D), and P/N 50 (7E). The transfection efficiencies for various P/N ratios are summarized based on the fluorescent cells in four independent field areas (FIG. 7F). The transfection experiments with various P/N ratio resulted that pDNA complexes of Silk6mer-30lys (P/N=10) demonstrated a highest percentage (14%±3%) of GFP-positive cells among the different complexes. The transfection efficiency based on the GFP-positive cells decreased in the following order: P/N=10, 25, 50, 5, and 2.5. Hence, further experiments to the DNA polylysine-silk complexes were prepared at a P/N ratio of 10.

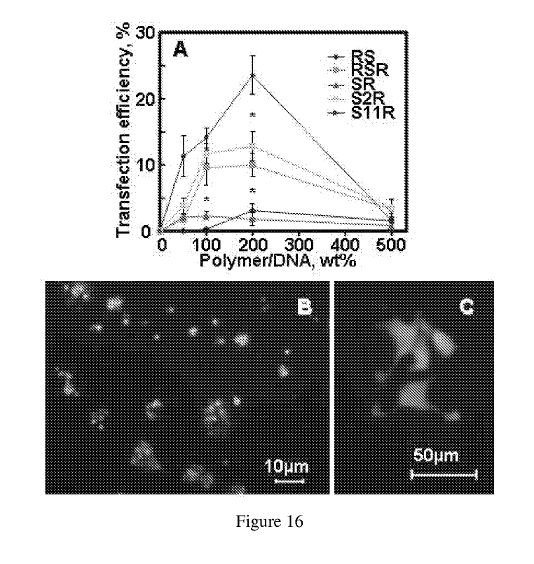

FIG. 16A shows the transfection efficiencies for DNA complexes of four recombinant RGD-silks with P/N ratios ranging from 50 to 500 based on the fluorescent cells in three independent field areas. FIG. 16B demonstrates a fluorescence microscopy image of cells incubated on the silk films containing pDNA complexes of 11RS prepared at P/N 200, which demonstrated the highest transfection efficiency among these samples. Also, pDNA complexes of the samples prepared at P/N 100 or 200 demonstrated a highest percentage of GFP-positive cells among the different P/N ratios. The pDNA complexes of 11RS exhibited higher transfection efficiency (24±3%) in comparison to RS, RSR, SR and SR2 (3±1, 10±2, 2±1, and 13±2%) in P/N 200. The significant difference was recognized between not only 11RS and RSR but also RSR and RS at P/N 200. Therefore, the relative order of the transfection efficiency at P/N 200 decreased as follows: 11RS>S2R≈RSR>RS≈SR, indicating that the transfection efficiency was strongly dependent on the number of RGD cell-binding motif.

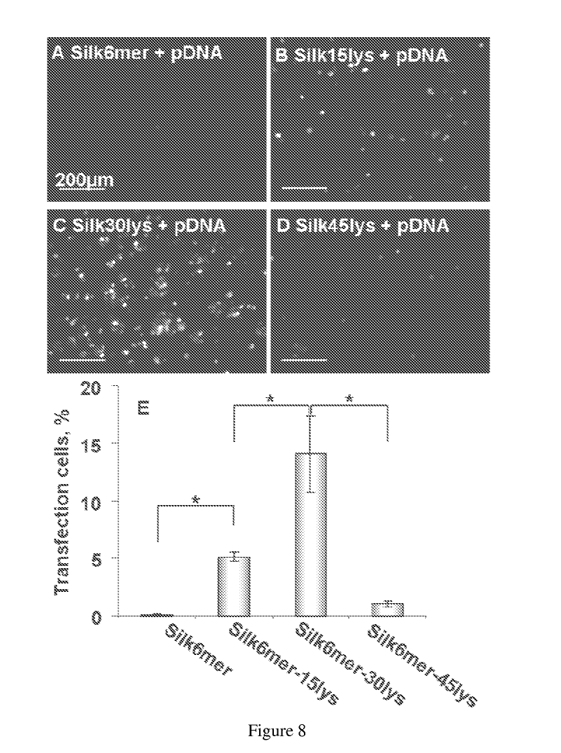

FIG. 8 shows the fluorescence microscopy images of cells incubated on the silk film containing pDNA complexes with the P/N ratio of 10 for Silk6mer (8A), Silk6mer-15lys (8B), Silk6mer-30lys (8C), and Silk6mer-45lys (8D). FIG. 8E shows the efficiency ratios of transfection determined as described above by counting GFP positive cells. The pDNA complexes of Silk6mer-30lys exhibited the highest transfection efficiency (14%±3%) among the four samples, whereas the mixture of Silk6mer and pDNA failed to show effective transfection (0.4%±0.1%). The relative order of the transfection efficiency decreased as follows: Silk6mer-30lys, Silk6mer-15lys, Silk6mer-45lys, and Silk6mer.

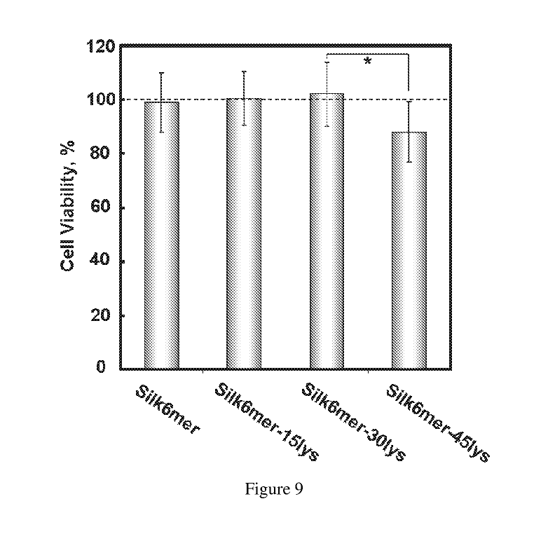

Cytotoxicity of DNA complexes with the P/N ratio of 10 for Silk6mer, Silk6mer-15lys, Silk6mer-30lys, and Silk6mer-45lys was measured using the MTT assay. FIG. 9 shows that the complexes of Silk6mer, Silk6mer-15lys, and Silk6mer-30lys exhibited no toxicity to HEK cells at the concentrations used in the transfection experiments (0.76 mg/ml). The DNA complexes of Silk6mer-45lys showed 88%±11% of cell viability, which was significantly different and lower in comparison with the other recombinant silk complexes. Cytotoxicity of DNA complexes with the P/N ratio of 500 for RS, RSR, SR and S2R was also measured using the MTT assay. FIG. 15 shows that the complexes of all samples exhibited no cytotoxicity to HEK cells at the highest concentration used in the transfection experiments (1.9 mg/mL).

The embodiments of the present invention provide for novel complexes of recombinant silk molecules with nucleic acid delivery. Eight types of recombinant silks were cloned, expressed, and purified from E. coli. The DNA complex of Silk6mer without the lysine sequence did not form globular particles based on AFM analysis (FIG. 4C). Additionally, agarose gel electrophoresis showed free DNA when mixed with the Silk6mer molecules. On the other hand, according to the electrophoresis experiments (FIG. 5A) and the AFM images (FIG. 4), globular complexes of DNA with recombinant silks containing polylysine were formed, suggesting that the polylysine sequence might be necessary to form globular nano-sized ion complexes of silk molecules with DNA. The diameter and size distribution of the DNA complexes increased with an increase in the molecular weight of polylysine sequence and the P/N ratio (Table 2 and FIG. 17). In the case of Silk6mer-lys45 or complexes prepared at P/N=25 or 50, relatively high positive charges of the recombinant silk produced larger and more widely-distributed complexes. Part of the DNA was released from the complexes during electrophoresis (lanes 3, 4, 5 in FIG. 5), implying that the DNA might be packed in the interior of the globular complexes as previously reported (Blessing et al., 95 P.N.A.S. USA 1427-31 (1998); Masotti et al., 19 Nanotech. 55302 (2008)), but also on the surface, although further studies may clarify this issue.

Another embodiment of the present invention provides for compositions comprising recombinant silk that contain cell-binding motifs complexed nucleic acid for nucleic acid delivery. Four types of recombinant RGD-silks, RS, RSR, SR, and S2R, were cloned, expressed, and purified from E. coli. Globular nano-sized ion complexes of silk molecules containing 30 lysines sequence with pDNA were formed, and the average sizes were less than 80 nm, such as 32, 72, 68, and 59 nm according to the electrophoresis experiments (FIG. 14A), the AFM images, and DLS measurements (FIG. 13). The diameter of the DNA complexes decreased with an increase in the P/N ratio (FIG. 13A), suggesting the sizes of the complexes can be controlled by the P/N ratio. Also, no DNA was released from the complexes during the electrophoresis, indicating that DNA was completely packed in the globular complexes as compared to other experiments in which some DNA was released from the complexes during electrophoresis.

Silk films containing the plasmid DNA complexes on the surface was prepared (FIG. 6A). Comparison of the height of complexes before and after deposition on the films (FIGS. 4C and 6B) supported that the DNA complexes were half-buried and immobilized on the surface of silk film, likely in part due to the partial local solubilization of the surface by the HFIP prior to evaporation. Additionally, silk-silk (protein-protein) hydrophobic interactions between the silk in the DNA complexes and the surfaces of silk biomaterial films supports the immobilization of the DNA complexes.

The transfection experiments with the complexes containing the GFP gene into HEK cells revealed that the DNA complex of Silk6mer-30lys prepared at P/N 10, which was 380 nm in diameter by DLS, was the most efficient complex of the Silk6mer and polylysine block copolymers (FIGS. 7 and 8). These findings, with regard to the variations in efficiency, might be related to particle size as described previously. Rejman et al., 377 Biochem. J. 159-69 (2004); Ross & Hui, 6 Gene Ther. 651-59 (1999); Almofti et al., 20 Mol. Membr. Biol. 35-43 (2003). In particular, it has been reported the particles of less than 200 nm in diameter are almost exclusively internalized, while particles of 500 nm in diameter are not, suggesting the size of particles for nucleic acid delivery are critical. Rejman et al., 2004; Ross & Hui, 1999; Almofti et al., 2003. Without being bound by theory, the diameters of pDNA complex of Silk6mer-45lys (P/N 10, 590 nm) and the complexes prepared at P/N>10 (more than 400 nm in diameter) might be too large to be transfected into the cells. Also, the relatively high positive charges of the complexes due to higher molecular weight of polylysine and P/N ratio might result in the formation of disordered aggregates in solution and induce cytotoxicity, thus reducing transfection efficiency as reported in some literature. Ogris et al., 1999; Oupicky et al., 1999; Sato et al., 22 Biomats. 2075-80 (2001); Thanou et al., 23 Biomats. 153-59 (2002). In this respect, the higher positive charge of the Silk6mer-45lys showed lower cell viability in comparison with the other complexes (FIG. 9), and also reduced the transfection efficiency (FIG. 8). A particular embodiment comprising the pDNA complex of Silk6mer-30lys prepared at P/N of 10 was feasible for nucleic acid delivery.

The transfection experiments with the silk-RGD complexes containing the GFP gene into HEK cells revealed that the DNA complex of S2R prepared at P/N 200, which was positively charged (18 mV zeta potential) and 121 nm in diameter by DLS, was the most efficient complex of the recombinant silks in this study (FIG. 16). S2R and RSR showed almost same transfection efficiency with various P/N ratios, and there was no significant difference. On the other hand, RS and SR, which contained only one RGD peptide, demonstrated lower transfection efficiency in comparison to S2R and RSR, suggesting two important topics as follows. One is that the transfection efficiency was strongly dependent on the number of RGD cell-binding motif. The other is that position of RGD motif, at N-terminus or C-terminus, might not influence on the transfection efficiency of the nucleic acid complexes of recombinant silks. In other words, the recombinant silk molecules in nucleic acid complexes were considered to be randomly assembled with DNA and RGD residues existed on the surface of the complexes as shown in FIG. 10. The number of RGD peptides on the surface of the complexes should therefore be proportional to the number of RGD residues in the recombinant silk, resulting that RSR and S2R, which contain dimer of RGD, showed higher cell-binding ability and transfection efficiency compared to RS and SR. Thus, other functional peptides can be added into any position of amino acid sequences in order to construct alternative novel proteins as nucleic acid/nucleic acid vectors.

RGD residues have been used as a ligand to enhance cell-binding function and cell transfection efficiency of gene vectors. In particular, polymer-based gene vectors to contain RGD sequences were studied by several groups. Oba, 2006; Kim, 2005; Connelly, 2007; Renigunta, 2006. Sun, Biomats. (2008); Moore, Molecular Pharmaceutics (2008); Oba, Bioconjugate Chem. (2007); Ishikawa, Bioconjugate Chem. (2008); Quinn, Mol. Ther. (2009); Singh, Gene Ther. (2003). Complexes of poly(ethyleneimine) (PEI) and RGD peptides showed higher transfection efficiency to HEK cells in comparison with only PEI molecules, however, the cytotoxicity of the complexes of PEI and RGD peptides was approximately 50% at the used concentration of 400 μg/mL. Sun, 2008. Poly(ethylene glycol) (PEG)-based vector demonstrates almost no cytotoxicity to HEK cells, and also exhibits comparable transfection efficiency to PEI. Moore, 2008. An addition of RGD peptides into platform chemical synthetic polymer, however, needs multi-steps of chemical reactions. Also, chemical synthetic polymer has distribution of molecular weight, implying their heterogeneous complexes with DNA.

Cell-penetrating and cell membrane-destabilizing peptides (CPPs) are defined as short peptides that efficiently penetrate cellular lipid bilayers or destabilize cellular membranes. Therefore, CPPs are useful candidates for new nonviral nucleic acid vectors. The CPP internalization mechanism was reported as a caveolae, clathrin-dependent endocytosis and macropinocytosis. Järver et al.,35 Biochem. Soc. Trans. 770-74 (2007); Richard et al., 278 J. Biol. Chem. 585-90 (2003); Ferrari et al., 8 Mol. Ther. 284-94 (2003); Holm et al., 1 Nat. Protoc. 1001-05 (2006); Lundberg & Langel, 12 Intl. J. Pept. Res. Ther. 105-14 (2006). The cellular uptake has also been reported to be independent of endocytotic pathways and occur through transient pore formation. Vivès et al., 1786 Biochim. Biophys. Acta. 126-38 (2008); Deshayes et al., 1667 Biochim. Biophys. Acta. 141-47 (2004); Deshayes, 43 Biochem. 1449-57 (2004); El-Andaloussi et al., 8 J. Gene. Med. 1262-73 (2006); Abes et al., 35 Biochem. Soc. Trans. 53-55 (2007). Additionally, it has been suggested that CPPs may simultaneously utilize different mechanisms of endocytosis and uptake occurs by an additional rapid translocation process. Duchardt et al., 8 Traffic 848-66 (2007). An addition of CPPs peptides into platform chemical synthetic polymer may enhance the efficiency of nucleic acid delivery systems; however, there has been no recombinant proteins that combine CPPs and the other functional sequences for non-viral nucleic acid delivery.

The present invention provides for recombinant silks synthesized using recombinant DNA techniques and an E. coli system, which is one-step synthesis. Moreover, in contrast to synthetic polymer the recombinant silk proteins demonstrate no distribution of molecular weight, which helps in preparing homogeneous nucleic acid complexes with the proteins. Also, the DNA complexes of recombinant silks showed no cytotoxicity to HEK cell at the highest concentration used in the transfection experiments (1.9 mg/mL), and also exhibited comparable transfection efficiency (13%±2%) in comparison to PEI (15%-40%). Fischer et al., 16 Pharm. Res. 1273-79 (1999); Ahn et al., 80 J. Controlled Release 273-82 (2002); Godbey, Gene Ther. (1999).

Further, the recombinant silks can be added any number of any peptides in expected positions of silk molecules, if the corresponding plasmid is constructed. In this respect, this recombinant silk-base nucleic acid delivery system is superior to general synthetic polymer-based nucleic acid delivery systems, because the synthetic polymer-based system has a limitation of molecular weight of additional peptides. In order to further enhance the efficiency and specificity of nucleic acid delivery, the recombinant silks prepared herein can be further modified with multi functional peptides, such as for cell-penetration and tumor-homing peptides. Elmquist et al., 269 Exp. Cell Res. 237-44 (2001); Rittner et al., 5 Mol. Ther. 104-14 (2002); Järver et al., 35 Biochem. Soc. Trans. 770-74 (2007); Mäkelä et al., 80 J. Virol. 80:6603-11 (2006); Laakkonen et al., 8 Nat. Med. 751-55 (2002); Porkka et al., 99 P.N.A.S. 7444-49 (2002); Christian et al., 163 J. Cell Biol. 871-78 (2003); Laakkonen et al., 100 P.N.A.S. USA 9381-86 (2004); Pilch et al., 103 P.N.A.S. 2800-04 (2006).

In particular, one of the highest transfection efficiencies of pDNA complexes with cell-penetrating peptides was reported to be approximately 45-fold higher in comparison to the pDNA complex of PEI at low DNA concentration (125 ng/mL) and without the specific penetrating peptide. Rittner et al., 2002. Thus, the addition of such peptides as an approach to enhance transfection efficiency and selectivity. Hence, the recombinant silk modified to contain RGD or polylysine can be a new platform polymer, like PEG, for nucleic acid delivery

Chemical synthetic polymer-based nonviral DNA delivery systems have been improved in effectiveness and in terms of biocompatible delivery over the last decade. Polyethylenimine (PEI) has become the standard in many in vitro and in vivo applications for DNA delivery with respect to transfection efficiency, DNA protection, cell-binding, and endosomal release. Blessing et al., 95 P.N.A.S. 1427-31 (1998); Kataoka et al., 47 Adv. Drug Deliv. Rev. 113-31 (2001); Schaffert & Wagner, 15 Gene Ther. 1131-38 (2008); Luten et al., 126 J. Control Release 97-110 (2008); Feng et al., 50 Biotechnol. Appl. Biochem. 121-32 (2008). One of the highest transfection efficiencies of PEI/DNA complexes to HEK cells was reported to be approximately 75%, obtained using the complex at a PEI/DNA ratio of 5:1 (w/w) after incubation of 54 hours. Feng et al., 2008. Nevertheless, it is necessary to improve synthetic polymer-based nucleic acid delivery systems in cytotoxicity, and specific-delivery properties and targeting. Fischer et al., 16 Pharm. Res. 1273-79 (1999). Cell viability from PEI was reported to be below 50% at concentrations of 0.1 mg/mL for 24 hours (id.), whereas the present bioengineered silk-based nucleic acid delivery system using the Silk6mer-30lys demonstrated no cytotoxicity for 48 hours as shown in FIG. 9. The best transfection efficiency (14±3%) in this study was lower in comparison with the PEI system. Blessing et al., 1998; Kataoka et al., 2001; Schaffert & Wagner, 2008; Luten et al., 2008; Feng et al., 2008; Fischer et al., 1999. Perhaps the pDNA complex of Silk6mer-30lys of a smaller, viral particle size, and positively charged may interact more readily with cell surfaces. Zauner et al., 1998; Blessing et al., 1998; Kataoka et al., 2001. In order to enhance the effective and specific delivery, the silk and polylysine block copolymers prepared herein can be further modified with functional peptides, such as for cell-penetration, cell-binding, and tumor-homing, through the use of genetic engineering. Järver et al., 35 Biochem. Soc'y Trans. 770-74 (2007); Rittner et al., 5 Mol Ther. 104-14 (2002); Laakkonen et al., 101 P.N.A.S. 9381-86 (2004); Laakkonen et al., 1131 Ann. NY Acad. Sci. 37-43 (2008).

In particular, one of the highest transfection efficiencies of DNA complexes with cell-penetrating peptides was reported to be approximately 45-fold higher in comparison to the DNA complex of PEI at low DNA concentration (125 ng/mL) and without the specific penetrating peptide. Rittner et al., 2002. Thus, the system may be improved by optional addition of such peptides as an approach to enhance transfection efficiency. Nevertheless, the recombinant silk modified to contain the polylysine sequence has the potential to be effective, specific, biodegradable, and completely biocompatible nucleic acid delivery system, even though the present transfection efficiency of silk-based delivery system is lower than the other competitors.

Furthermore, the present invention provides for a method of directly transfecting the nucleic acid complexes immobilized on the surface of silk films to cells. This is the first report of nucleic acid transfection from a polymeric single-layer film. Hence, the silk-based complexes with nucleic acid (such as DNA), which could be transfected in cells and were able to be adsorbed on the surface of the silk film, such that this new nucleic acid delivery system can be applied not only to silk films but also to other silk-based biomaterials for nucleic acid delivery. The versatility in both design and application of these new novel bioengineered silk protein delivery systems for nucleic acid suggests future utility in many nucleic acid delivery applications.

The cationic recombinant silk proteins provide a number of advantages as nucleic acid delivery systems, when compared with polylysine alone. Polylysine can form pDNA complexes for nucleic acid delivery and offer features such as biodegradability, low-cytotoxicity, and flexibility regarding the size of the pDNA complex (15 nm to 150 nm in diameter). Zauner et al., 1998. pDNA complexes with polylysine, however, need improved in-vivo stability against enzymes that degrade the pDNA. Id. Further, polylysine heterogeneity with respect to molecular weight presents challenges in the preparation of homogeneously sized pDNA complexes. In contrast, recombinant silk proteins can enhance in-vivo stability of pDNA complexes. Choi et al., 10 Bioconj. Chem. 62-65 (1999); Gottschalk et al., 3 Gene Ther. 448-57 (1996). Further, the homogeneous molecular weight of the recombinant silk and polylysine system described herein provides monodisperse polymeric components that can provide improved control of the desired pDNA complexes by further refining the system. Additionally, the immobilization of pDNA complexes on the surface of films enhanced the internalization of pDNA by cells and promoted surface-mediated transfection. Segura et al., 13 Bioconj. Chem. 621-29 (2002); Shen et al., 3 Nat. Mats. 569-74 (2004); Park et al., 22 Langmuir 8478-84 (2006); Jewell & Lynn, 60 Adv. Drug Deliv. Rev. 979-99 (2008).

The present invention thus provides for compositions and methods for the transfection of nucleic acids in cells through biodegradable and biocompatible silk-based complexes. Recombinant silks modified to contain polylysine sequences were prepared and used to form globular complexes with nucleic acid polymers. Silk films containing the nucleic acid complexes on their surface were also prepared, and direct transfection of DNA complexes immobilized on the surface of silk films to HEK cells was carried out successfully.

Some embodiments of the present invention also provide for the novel transfection of nucleic acids to cells via biodegradable and biocompatible recombinant silks modified to contain RGD cell-binding motifs. Recombinant silks modified to contain polylysine and RGD residues were prepared and used to form globular complexes with pDNA. Transfection of the pDNA complexes to HEK cells was successfully carried out. The nucleic acid transfection experiments in HEK cells revealed that the pDNA complex of S2R prepared at P/N 200, which were approximately 100 nm in diameter by DLS with a zeta potential of around 10mV, was the complex with the highest efficiency (13±2%) of all the recombinant silks examined. The transfection efficiency was strongly dependent on the number of RGD cell-binding motif. Further, the position of RGD motif, at N-terminus or C-terminus of the recombinant silks, did not influence on the transfection efficiency of the pDNA complexes. Thus, recombinant silks containing RGD or polylysine residues have demonstrated feasibility for application to silk-based materials for nucleic acid delivery.

Some embodiments of the present invention provide for novel methods and composition as nucleic acid delivery vectors to enhance the transfection efficiency of nucleic acids by adding CPPs into silk-based cationic block copolymer systems containing recombinant silk-polylysine. In one embodiment, the CPP used was ppTG1, which shows a high transfection efficiency of pDNA complexes with CPPs. Rittner et al., 5 Mol. Ther. 104-14 (2002). However, the DNase resistance and stability of the pDNA complexes with ppTG1 peptides have not been investigated, perhaps because the peptides contain no functional sequence, like a sequence of silk, to protect their incorporated nucleic acids from nucleic acid-degrading enzymes. The recombinant silk protein incorporating CPPs (e.g., ppTG1) complexed with nucleic acids, however, has improved efficiency of nucleic acid delivery, and present increased stability and resistance to DNase. In one embodiment, complexes of these silk-based block copolymers with pDNA were prepared for in vitro nucleic acid delivery to HEK and MDA-MB-435 cells, and characterized by agarose gel electrophoresis, zeta potentialmeter, atomic force microscopy (AFM), and dynamic light scattering (DLS). The polymer properties of silks in terms of self-assembly, robust mechanical properties and controllable rates of degradation, in combination with tailored ionic complexation with plasmid DNA and the cell-penetrating function reported here, provide a new family of vehicles for the nucleic acid delivery and optimization.

In a particular embodiment, a novel complexes of recombinant silk proteins with CPPs for nucleic acid delivery was designed, and how CPPs enhanced transfection efficiency was investigated. The recombinant silk proteins, Silk-polylysine-ppTG1 monomer and dimer, were prepared using E. coli, and then formed in complexes with pDNA (Table 5). The average diameters of the pDNA complexes characterized were the same as designed, and are appropriate for nucleic acid delivery, according to the literature. Yan et al., 276 J. Biol. Chem. 8500-06 (2001); Thomas & Smart, 51 J. Pharmacol. Toxicol. Meth. 187-200 (2005). The pDNA complexes prepared at an N/P (i.e., the ratio of numbers of amines to phosphate in DNA) of about 2 to about 5 demonstrated useful transfection efficiency. The pDNA complexes before and after methanol treatment were capable of protecting the incorporated pDNA from DNase I, as shown in FIG. 26, which implies the recombinant silk protein may be of protective or on the outside surface of the complexes and can prevent DNase from accessing to the pDNA. In summary, the pDNA complexes of the recombinant silk containing CPPs are biodegradable, biocompatible and also provide resistance to DNase, an advantage for a non-viral nucleic acid delivery carrier.

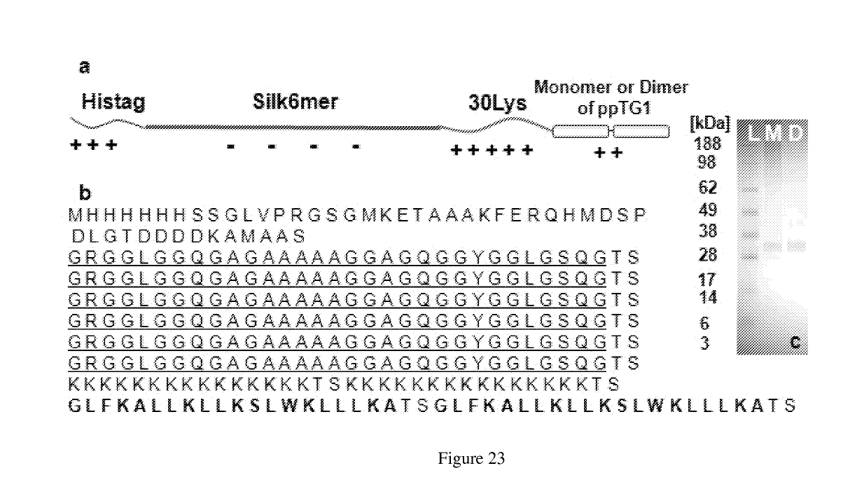

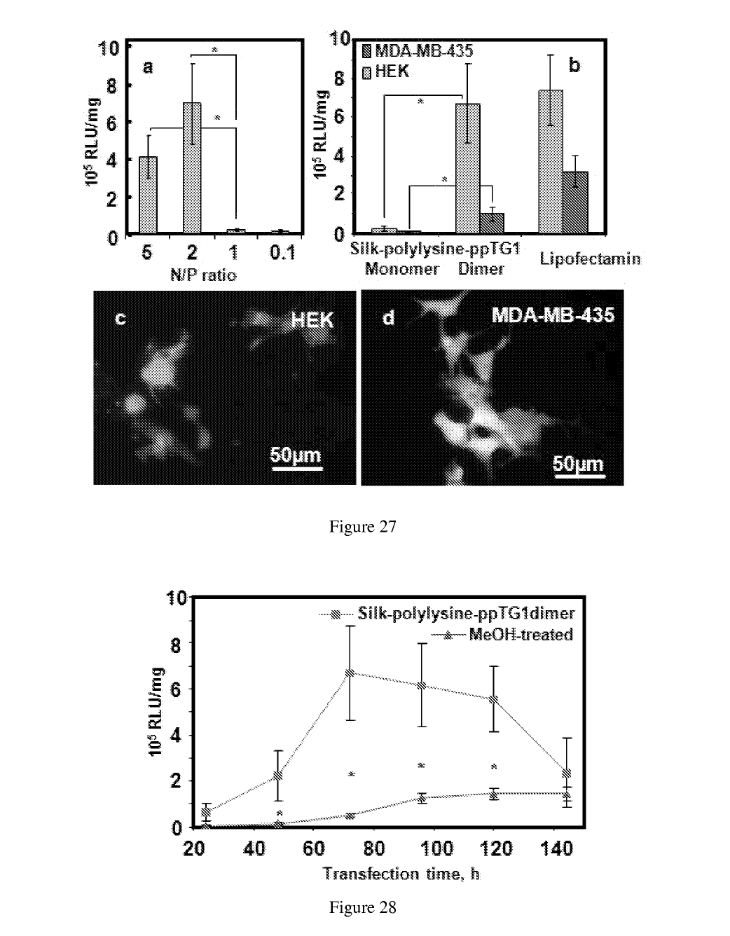

Silk-polylysine-ppTG1 monomer did not appear to provide substantial transfection efficiency to the two tested cells (HEK cells and MDA-MB-435 cells). Silk-polylysine-ppTG1 dimer, however, demonstrated 25-fold higher transfection efficiency than the monomeric version and a similar level of efficiency to HEK cells as Lipofectamine 2000, as shown in FIG. 27B. Without being bound by theory, this enhancement of transfection efficiency by the addition of dimeric ppTG1 sequence vs. the monomeric version, may demonstrate the importance of this peptide in terms of cell access. ppTG1 peptide was reported to have functions to bind pDNA as well as to destabilize cell membranes. Rittner et al., 5 Mol. Ther. 104-14 (2002). Moreover, in in-vitro transfection assays, ppTG1 was reported to show a high transfection efficiency, approximately 45-fold higher in comparison to the pDNA complex of polyethyleneimine, at low N/P ratio and low concentration (125 ng/mL), different from the other CPPs. Id. Transfection experiments in the presence of Bafilomycin A, which is a specific inhibitor of the vacuolar proton pump (Sun et al., 29 Biomats. (2008) 4356-65 (2008)), suggested cellular uptake of pDNA complexes of the ppTG1 peptides may be through the cytoplasmic membrane or via endocytosis (Rittner et al., 2002). Moreover, molecular modeling of ppTG1 suggested that all lysines may segregate on the same side when the structure is an alpha-helix, rendering the helix amphipathic. The lysine residues could then interact with the negative charges of DNA, because the mean distance between two amine groups is similar to the mean distance between two phosphates on the DNA strands. El-Andaloussi et al., 8 J. Gene. Med. 1262-73 (2006); Abes et al., 35 Biochem. Soc. Trans. 53-55 (2007); Duchardt et al., 8 Traffic 848-66 (2007); Rittner et al., 2002. In spite of previous studies on ppTG1, the function of the dimeric ppTG1, however, has not been reported previously. In the present system comprising a nucleic acid complexed with recombinant silk-polylysine-ppTG1, the polylysine as well as ppTG1 sequences may interact with nucleic acid and the nucleic acid complexes can be transferred into cells through the cell membrane. It appeared that Silk-polylysine-ppTG1 dimer formed the expected secondary structure to destabilize the cell membrane via ppTG1 sequences and showed a significantly higher transfection efficiency (FIG. 27B) than Silk-polylysine-ppTG1 monomer, where the polylysine sequence is proximal to the ppTG1 sequence (FIGS. 23A and 23B).

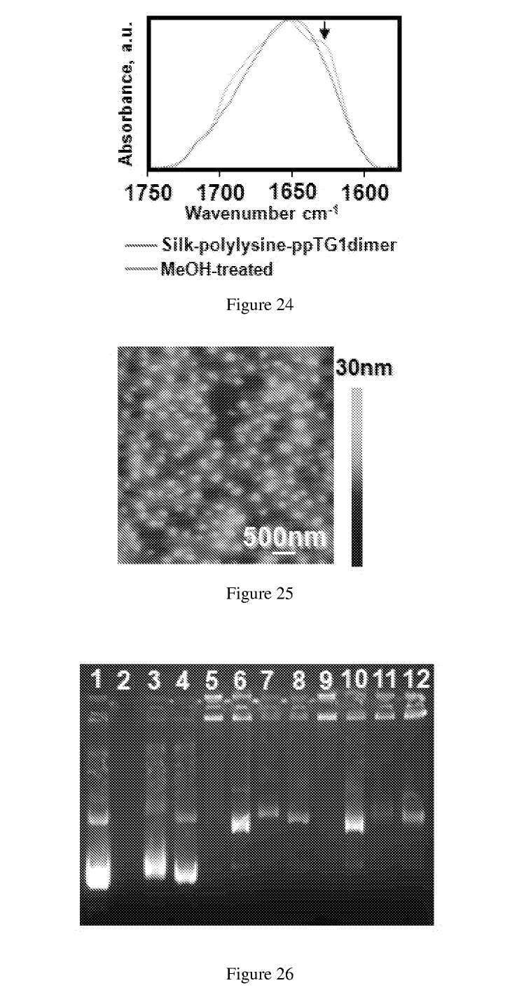

The methanol-treated pDNA complexes, which contain the beta-sheet structure based on FTIR-ATR measurements (FIG. 24), showed a different transfection behavior from the complexes of Silk-polylysine-ppTG1 dimer without methanol treatment (FIG. 28). This result indicated a way to achieve more sustained or constant release of pDNA from the methanol-treated silk-based complexes vs the nonmethanol treated system. This result is because the beta-sheet structure of silk sequences induced by the methanol treatment decreases the enzymatic degradation rate of the complexes. Additionally, the methanol-treated pDNA complexes after the enzymatic treatment by alpha-chymotrypsin released smaller amounts of free pDNA in comparison with treatment by protease XIV (FIG. 26, lanes 11,12). Alpha-chymotrypsin hydrolyzes non-crystalline silk fibroins, whereas protease XIV digests not only non-crystalline but also beta-sheet (crystalline) silk domains. Numata et al., 2010; Bowman et al., 1988; Huang et al., 2003. Therefore, the secondary structure of the silk sequence, such as beta-sheet structure content, can be used to control the release profile of pDNA from these complexes due to different enzymatic degradation rates polymer, like PEG, for nucleic acid delivery, but with tremendous versatility in design and function.

The methods and compositions provided herein can also be used to deliver nucleic acids to cells for the purpose of reprogramming a cell. For example, a nucleic acid encoding a reprogramming factor can be delivered to a cell to produce an induced pluripotent stem (IPS) cell. The term “re-programming” as used herein refers to the process of altering the differentiated state of a terminally-differentiated somatic cell to a pluripotent phenotype.

As used herein, the term “reprogramming factor” refers to a nucleic acid that promotes or contributes to cell reprogramming to an induced pluripotent stem cell phenotype, e.g., in vitro. A reprogramming factor is added exogenously or ectopically to the cell using the methods of nucleic acid delivery described herein. The reprogramming factor is preferably, but not necessarily, from the same species as the cell being reprogrammed, i.e., human reprogramming factors for human cells. Non-limiting examples of reprogramming factors of interest for reprogramming somatic cells to pluripotency in vitro are Oct¾ (Pouf51), Sox1, Sox2, Sox3, Sox 15, Sox 18, NANOG, Klf1, Klf2, Klf4, Klf5, c-Myc, 1-Myc, n-Myc and LIN28, and any gene/protein or molecule that can substitute for one or more of these in a method of reprogramming somatic cells in vitro. “Reprogramming to a pluripotent state in vitro” is used herein to refer to in vitro reprogramming methods that do not require, and typically do not include, nuclear or cytoplasmic transfer or cell fusion, e.g., with oocytes, embryos, germ cells, or pluripotent cells.

To confirm the induction of pluripotent stem cells, isolated clones can be tested for the expression of a stem cell marker. Such expression identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. Methods for detecting the expression of such markers can include, for example, RT-PCR and immunological methods that detect the presence of the encoded polypeptides.

The pluripotent stem cell character of the isolated cells can be confirmed by any of a number of tests evaluating the expression of embryonic stem cell markers and the ability to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers further indicates that the cells are pluripotent stem cells.

### EXAMPLES

### Example 1

### Design and Cloning of Recombinant Silk-Polylysine Molecule

The spider silk repeat unit was selected based on the consensus repeat (SGRGGLGGQGAGAAAAAGGAGQGGYGGLGSQGT) derived from the native sequence of the dragline protein MaSp1 sequence from the spider Nephila clavipes (Accession P19837). The 6mer containing six contiguous copies of this repeat was developed through the transfer of cloned inserts to pET-30a, which had been modified with a linker carrying the restriction sites NheI and SpeI according to previously published procedures. Prince et al., 34 Biochem. 10879-85 (1995). The sequences of the synthetic oligonucleotides encoding 15 lysine residues were as follows:

| | |
|---|---|
| Lys-a: | 5′-CTAGCAAGAAAAAGAAAAAAAAGAAAAAAAAGAAAAAGA |
| | AAAAAAAGAAAA-3′, |
| Lys-b: | 5′-CTAGTTTTCTTTTTTTTCTTTTTCTTTTTTTTCTTTTTT |
| | TTCTTTTTCTTG-3′. |

The restriction sites for NheI and SpeI are italicized. Lys-a and Lys-b are complementary oligonucleotides which were annealed to form double stranded DNA. The newly formed double stranded DNA was then ligated and multimerized to form the monomer (15 lysines), dimer (30 lysines), and trimer (45 lysines). The double stranded DNAs of polylysine sequences were ligated into pET30-6mer to generate pET30-6mer-polylysine by DNA ligase (New England Biolabs Inc., Ipswich, Mass.).

### Example 2

### Recombinant Silk-Polylysine Protein Expression and Purification

The constructs pET30-6mer (control), pET30-6mer-15lysines, pET30-6mer-30lysines, and pET30-6mer-45lysines were used to transform the E. coli strain RY-3041, a mutant strain defective in the production of the SlyD protein, and protein expression carried out by methods reported previously. Huang et al., 278 J. Biol. Chem. 46117-23 (2003); Yan et al., 276 J. Biol. Chem. 8500-06 (2001). Briefly, cells were cultivated in LB broth containing kanamycin (50 μg/ml) at 37° C. Protein expression was induced by the addition of 0.5 mM IPTG (Sigma-Aldrich, St. Louis, Mo.) when the OD600 nm reached 0.6. After approximately 4 hr of protein expression, cells were harvested by centrifugation at 13,000 g. The cell pellets were resuspended in denaturing buffer (100 mM NaH₂PO₄, 10 mM Tris HCl, 8 M urea, pH 8.0) and lysed by stiffing for 12 hr followed by centrifugation at 13,000 rcf at 4° C. for 30 min. His-tag purification of the proteins was performed by addition of Ni-NTA agarose resin (Qiagen, Valencia, Calif.) and 20 mM imidazole to the supernatant (batch purification) under denaturing conditions. After washing the column with denaturing buffer at pH 6.3, the proteins were eluted with denaturing buffer at pH 4.5 (without imidazole). SDS-polyacrylamide gel electrophoresis (PAGE) was performed using 4-12% precast NuPage Bis-Tris gels (Invitrogen, Carlsbad, Calif.). The gel was stained with Colloidal blue (Invitrogen). Purified samples were extensively dialyzed against Milli-Q water. For dialysis, Slide-A-Lyzer Cassettes (Pierce, Rockford, Ill.) with MWCO of 3,500 were used. The dialyzed samples were dissolved in 1 mL of hexafluoroisopropanol (HFIP). The recombinant proteins were further characterized for sequence confirmation at the Tufts University Core Facility by LC/MS/MS analysis.

### Example 3

### Preparation and Characterization of the pDNA Complexed with the Recombinant Silk-Polylysine

pDNA encoding GFP (EGFP, 7,650 bp) was amplified in competent DH5α E. coli (Invitrogen) and purified using EndoFree Plasmid Maxi Kits (Qiagen, Hilden, Germany). The DNA concentration was determined by absorbance at 260 nm. To prepare the complexes of the recombinant silk proteins with pDNA, an HFIP solution containing silk protein (10 mg/mL) was mixed with the pDNA solution (370 μg/mL) at various P/N ratios. Here, P/N ratio refers to the molar ratio of the recombinant silk to nucleotides in pDNA. The mixture of recombinant silk and pDNA was incubated at room temperature (˜20° C.) overnight prior to characterization. The pDNA complexes were characterized by agarose gel electrophoresis, dynamic light scattering (DLS, Brookhaven Instruments Corporation, Holtsville, N.Y.) and atomic force microscope (AFM, Dimension V, Veeco Instruments Inc., Plainview, N.Y.). For agarose gel electrophoresis, 10 μL of each sample was mixed with loading buffer and analyzed on 1% agarose gel containing ethidium bromide (TAE buffer, 100 V, 60 min). DLS was performed using a 532 nm laser at 37° C. with a scattering angle of 90°, and the particle size and its distribution were analyzed using Dynamic Light Scattering software (Brookhaven Instruments Corp.). The pDNA silk complex solution (around 70 μL) was added to ultra pure water (450 μL) and then used as a sample for DLS measurement. AFM observations were performed in air at room temperature using a 200-250 μm long silicon cantilever with a spring constant of 2.8 N/m in tapping mode AFM. Calibration of the cantilever tip-convolution effect was carried out to obtain the true dimensions of objects by previously reported methods. Numata et al., 6 Macromol. Biosci. 41-50 (2006).

### Example 4

### Preparation of Films Containing pDNA Complexes

Silk fibroin was extracted from the cocoons from B. mori silkworm (Tajima Shoji Co., Yokohama, Japan) and silk solution (5 wt %) was prepared as previously described. Jin & Kaplan, 424 Nature 1057-61 (2003). The silk solution was cast in 24-multiwell and 96-multiwell plates, and silk films were obtained after evaporation of solvent, afterwards, the silk films were sterilized with ethanol solution (70%). To prepare the silk films containing the pDNA complexes, the pDNA silk complex solution (HFIP/water) was cast on the silk film and dried for at least 12 hr at room temperature to remove the solvent (HFIP/water). The silk films were washed with ultra pure water (DNAse, RNAse free, Invitrogen) to remove free pDNA before their use in cell transfection experiments.

### Example 5

### Cell Culture and Transfection

HEK cells (293 FT), which have served extensively as an expression tool for recombinant proteins, were used as a model cell line. See, e.g., Thomas & Smart, 51 J. Pharmacol. Toxicol. Methods 187-200 (2005). Cultures were grown to confluence using media consisting of DMEM, 10% FBS, 5% glutamine, 5% NEAA. The cultures were detached from their substrates using 0.25% trypsin (Invitrogen), and then replated on the pDNA silk complex-loaded silk films in the 24-multiwell plate at a density of 5,000 cells/cm² with 2.5 μL lipofectamine (Invitrogen). After incubation of the cells for 24 hr at 37° C., fluorescence images were obtained by fluorescence microscope (Leica Microsystems, Wetzlar, Germany) to evaluate GFP plasmid transfection. Expression results (n=4) were represented as the percentage of positive cells for GFP fluorescence relative to total cells counted.

For cell viability analysis, HEK cells (50,000 cells/well) were seeded into 96-well plates containing the pDNA complexes and cultured for 48 hr in the media (100 μL) used in the transfection experiment. Cytotoxicity to HEK cells of the pDNA complexes was characterized by a standard 3-(4,5-dimethylythiazol-2-yl)2,5-diphenyltetrazolium bromide) (MTT) assay (Promega, Madison, Wis.) according to the manufacturer's instructions (n=8).

### Example 6

### Design and Cloning of Silk Sequence Including RDG

The spider silk repeat unit was selected based on the consensus repeat (SGRGGLGGQGAGAAAAAGGAGQGGYGGLGSQGT) derived from the native sequence of the dragline protein MaSp1 sequence from the spider Nephila clavipes (Accession P19837). The Silk6mer-30lys containing six contiguous copies of this repeat and 30 lysines was developed through the transfer of cloned inserts to pET-30a, according to procedures published previously. Prince et al., 1995; Huang et al., 2003. The sequences of the synthetic oligonucleotides encoding RGD residues were as follows: RGD-a: 5′-CTAGCCGAGGCGACA-3′, RGD-b: 5′-CTAGTGTCGCCTCGG-3′. The restriction sites for NheI and SpeI are italicized. RGD-a and RGD-b are complementary oligonucleotides which were annealed to form double stranded DNA. The double stranded DNAs of RGD sequences were ligated into pET30-6mer-polylysine to generate five types of pET30-6mer-polylysine-RGD, as shown in FIG. 11, by DNA ligase (New England Biolabs Inc, Ipswich, Mass.).

### Example 7

### Recombinant Silk-RDG Protein Expression and Purification

The constructs pET30-RGD-6mer-30lysines, pET30-RGD-6mer-30lysines-RGD, pET30-6mer-30lysines-RGD, pET30-6mer-30lysines-2×RGD, and pET30-11×RGD-6mer-30lysines were used to transform the E. coli strains RY-3041, a mutant strain defective in the production of the SlyD protein, and protein expression carried out by methods reported previously. Huang et al., 278 J. Biol. Chem. 46117-23 (2003); Yan et al., 276 J. Biol. Chem. 8500-06 (2001). Briefly, cells were cultivated in LB broth containing kanamycin (50 μg/ml) at 37° C. Protein expression was induced by the addition of 1.0 mM IPTG (Sigma-Aldrich, St. Louis, Mo.) when the OD600 nm reached 0.6. After approximately 4 hr of protein expression, cells were harvested by centrifugation at 13,000 g. The cell pellets were resuspended in denaturing buffer (100 mM NaH₂PO₄, 10 mM Tris HCl, 8 M urea, pH 8.0) and lysed by stiffing for 12 hr followed by centrifugation at 13,000 g at 4° C. for 30 min. His-tag purification of the proteins was performed by addition of Ni-NTA agarose resin (Qiagen, Valencia, Calif.) and 20 mM imidazole to the supernatant (batch purification) under denaturing conditions. After washing the column with denaturing buffer at pH 6.3, the proteins were eluted with denaturing buffer at pH 4.5 (without imidazole). SDS-polyacrylamide gel electrophoresis (PAGE) was performed using 4%-12% precast NuPage Bis-Tris gels (Invitrogen, Carlsbad, Calif.). The gel was stained with Colloidal blue (Invitrogen, Carlsbad, Calif.). Purified samples were extensively dialyzed against Milli-Q water. For dialysis, Spectra/Por Biotech Cellulose Ester Dialysis Membranes with MWCO of 100-500 Da (Spectrum Laboratories Inc, Rancho Dominguez, Calif.) were used. The recombinant proteins were further characterized to confirm sequence and molecular weight at the Tufts University Core Facility by MALDI-TOF.

### Example 8

### Preparation and Characterization of the pDNA Encoding GFP Complexed with the Recombinant Silk-RGD.

Plasmid DNA (pDNA) encoding GFP (EGFP, 7,650 bp) was amplified in competent DH5α E. coli (Invitrogen) and purified using EndoFree Plasmid Maxi Kits (Qiagen, Hilden, Germany). The DNA concentration was determined by absorbance at 260 nm. To prepare the complexes of the recombinant silk proteins with pDNA, a solution containing silk protein (10 mg/mL) was mixed with the pDNA solution (370 μg/mL) at various P/N ratios. Here, P/N ratio refers to the weight ratio of the recombinant silk polymer to nucleotides in pDNA. The mixture of recombinant silk and pDNA was incubated at room temperature (˜20° C.) overnight prior to characterization. The pDNA complexes were characterized by agarose gel electrophoresis, zeta potentialmeter (Zetasizer Nano-ZS, Malvern Instruments Ltd, Worcestershire, UK), DLS (Brookhaven Instruments Corporation, Holtsville, N.Y.), and AFM (Dimension V, Veeco Instruments Inc., Plainview, N.Y.). For agarose gel electrophoresis, 10 μL of each sample was mixed with loading buffer and analyzed on 1% agarose gel containing ethidium bromide (TAE buffer, 100V, 60 min). Zeta potential and zeta deviation of samples were measured three times by zeta potential meter, and the average data were obtained using Dispersion Technology Software version 5.03 (Malvern Instruments Ltd). DLS was performed using a 532 nm laser at 37° C. with a scattering angle of 90°, and the particle size and its distribution were analyzed using Dynamic Light Scattering software (Brookhaven Instruments Corporation). The pDNA complex solution (around 70 μL) was added to ultra pure water (450 μL, Invitrogen) and then used as a sample for DLS measurement. The pDNA complex solution was cast on cleaved mica, and observed in air at room temperature using a 200-250 μm long silicon cantilever with a spring constant of 2.8 N/m in tapping mode AFM. Calibration of the cantilever tip-convolution effect was carried out to obtain the true dimensions of objects by previously reported methods. Numata et al., 6 Macromol. Biosci. 41-50 (2006).

### Example 9

### Cell Culture, Transfection, and Viability

HEK cells (293 FT), which have been extensively used as an expression tool for recombinant proteins, were used as a model cell line. Cultures were grown to confluence using media consisting of Dulbecco's Modified Eagle Medium (DMEM), 10% FBS, 5% glutamine, 5% Non-Essential Amino Acid (NEAA). The cultures were detached from their substrates using 0.25% trypsin (Invitrogen), and then replated on the films in the 96-multiwell plate at a density of 1500 cells/well. pDNA (1.2 μg) and recombinant silk (appropriate amount) complexes were added into each well. After incubation of the cells for 6 hr at 37° C., the media was exchanged to the media without pDNA complexes. After another incubation for 48 hr, fluorescence images were obtained by fluorescence microscope (Leica Microsystems, Wetzlar, Germany) to evaluate GFP plasmid transfection. Expression results (n=3) were represented as the percentage of positive cells for GFP fluorescence relative to total cells counted. For cell viability, HEK cells (50,000 cells/well) were seeded into the 96-wells plates containing the pDNA complexes and cultured for 48 hr in the media (100 μL) used in the transfection experiment. Cytotoxicity to HEK cells of the pDNA complexes was characterized by a standard 3-(4,5-dimethylythiazol-2-yl)2,5-diphenyltetrazolium bromide) (MTT) assay (Promega, Madison, Wis.) according to the manufacturer's instructions (n=8).

### Example 10

### Preparation and Characterization of the pDNA Encoding Luciferase Complexed with the Recombinant Silk-RGD

pDNA encoding Firefly Luciferase (7041 bp) was amplified in competent DH5α E. coli (Invitrogen) and purified using EndoFree Plasmid Maxi Kits (Qiagen, Hilden, Germany). The DNA concentration was determined by absorbance at 260 nm. To prepare the complexes of the recombinant silk proteins with pDNA, a solution containing silk protein (0.1 mg/mL) was mixed with the pDNA solution (370 μg/mL) at various N/P ratios (0.1 to 10). Here, N/P ratio refers to the ratio of number of the amines to phosphates in pDNA. The mixture of recombinant silk and pDNA was incubated at room temperature (˜20° C.) overnight to make sizes of the complexes homogeneous prior to characterization. The pDNA complexes were characterized by agarose gel electrophoresis, zeta nanosizer (Zetasizer Nano-ZS, Malvern Instruments Ltd, Worcestershire, UK), DLS (Brookhaven Instruments Corp., Holtsville, N.Y.) and AFM (Dimension V, Veeco Instruments Inc., Plainview, N.Y.). For agarose gel electrophoresis, 10 μL of each sample was mixed with loading buffer and analyzed on 1% agarose gels containing ethidium bromide (TAE buffer, 100 V, 60 min). Zeta potential and zeta deviation of samples were measured three times by zeta nanosizer, and the average data were obtained using Dispersion Technology Software version 5.03 (Malvern Instruments Ltd). DLS was performed using a 633 nm He—Ne laser at 25 ° C. with a scattering angle of 173°, and the particle size and distribution (PDI) were determined using Dispersion Technology Software version 5.03 (Malvern Instruments Ltd.). The pDNA complex solution was cast on cleaved mica, and observed in air at room temperature using a 200-250 μm long silicon cantilever with a spring constant of 2.8 N/m in tapping mode AFM. Calibration of the cantilever tip-convolution effect was carried out to obtain the true dimensions of objects by previously reported methods. Numata et al., 2006.

### Example 11

### Cell Culture, Transfection, and Viability

HeLa cells, which have been reported to express αᵥβ₃ and αᵥβ₅ integrins, and human embryonic kidney (HEK) cells (293 FT), which have been extensively used as a gene expression tool and reported to possess no αᵥβ₃ and a few αᵥβ₅ integrins, were used as a model cell line. Oba et al., 2007; Hu et al., 270 J. Biol. Chem. 26232-38 (1995); Simon et al., 272 J. Biol. Chem. 29380-89 (1997); Thomas & Smart, 2005. Cultures were grown to confluence using media consisting of DMEM, 10% FBS, 5% glutamine, 5% NEAA. The cultures were detached from their substrates using 0.25% trypsin (Invitrogen), and then replated on the films in the 24-multiwell plate at a density of 7000 cells/well. Media for transfection to HeLa and HEK cells were DMEM containing 10% FBS. pDNA (1.2 μg) and recombinant silk (appropriate amount) complexes were added into each well. After incubation of the cells for 6 hr at 37° C., the media was exchanged to the media without pDNA complexes. After another incubation for 48 hr, the Luciferase assay (Promega, Madison, Wis.) was performed (n=4) according to the manufactuer's protocol to evaluate the luciferase gene expression quantitatively. Briefly, the transfected cells were washed with PBS (Invitrogen) and lysed with Luciferase Cell Culture Lysis Regent (Promega). The lysate was mixed with Luciferase Assay Substrate and Luciferase Assay Buffer (Promega), and then the luciferase gene expression was evaluated based on the intensity of photoluminescence (the relative light unit) using luminescence microplate reader (Spectra MAX Gemini EM, Molecular Devices Corporation, Sunnyvale, Calif.). The amount of protein in each well was determined using BCA protein assay (Pierce Biotech., Rockford, Ill.), and then the relative light unit/weight of protein (RLU/mg) was obtained. Lipofectamine 2000 (Invitrogen) was used as a positive control vector in this experiment. For cell viability, HEK cells (5000 cells/well) were seeded into the 96-wells plates containing the pDNA complexes and cultured for 48 h in the media (100 μL) used in the transfection experiment. Cytotoxicity to HEK cells of the pDNA complexes was characterized by a standard 3-(4,5-dimethylythiazol 2-yl) 2,5-diphenyl tetrazolium bromide) (MTT) assay (Promega) according to the manufacturer's instructions (n=8).

### Statistical Analysis

The particle sizes on the silicon wafers were measured by AFM using a Research Nanoscope software version 7.30 (Veeco). The average value of 30 measurements was used. Statistical differences in particle sizes by AFM, cell transfection efficiency, and cell viability were determined by unpaired t-test with a two-tailed distribution and differences were considered statistically significant at p<0.05. The data in the AFM, cell transfection efficiency, and cell viability experiments are expressed as means±standard deviation.

### Example 12

### Confocal Laser Scanning Microscopy (CLSM)

pDNA was labeled with Cy5 using a Label IT Nucleic Acid Labeling Kit (Minis, Madison, Wis.), according to the manufacture procedure. HeLa cells were seeded on Glass Bottom Culture Dishes (MatTeK Corporation, Ashland, Mass.) and incubated overnight in 2 mL of DMEM. Complexes of the labeled pDNA (2.4 μg) with 11RS protein (N/P 2) were added into the wells. After incubation for 6 hr, the medium was replaced with fresh medium. After another incubation for 48 hr, the cells were washed with PBS twice and incubated with 300 nM 4′,6- diamidino-2-phenylindole (DAPI, Invitrogen) PBS solution for 10 min. The intracellular distributions of the pDNA complex labeled by Cy5 and the nuclei stained with DAPI were observed by CLSM (Leica Microsystems) at an excitation wavelength of 488 nm (Ar laser), 633 nm (He—Ne laser), and 710 nm (Mai Tai laser).

### Example 13

### Results and Discussion of the Nucleic Acid Delivery System Containing dRNA Encoding Luciferase Complexed with the Recombinant Silk-RGD and use Thereof for Cell Transfection

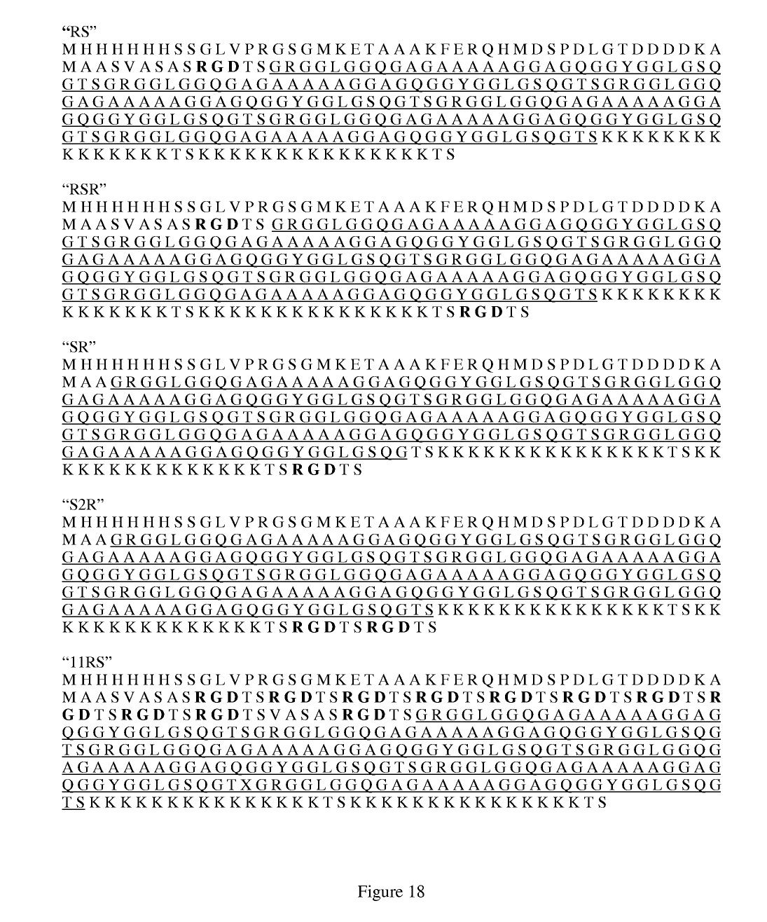

Expression and purification of silk protein including polylysine and RGD: The amino acid sequences of the five spider silk variants generated with polylysine and RGD cell-binding motifs (RS, RSR, SR, S2R, and 11RS) were generated, as shown in FIG. 11 and FIG. 18. Yields of the recombinant silk proteins were approximately 10 mg/L after purification and dialysis. The proteins before and after purification by Ni-NTA chromatography were analyzed by SDS-PAGE and stained with Colloidal blue to evaluate purity. RS, RSR, SR, S2R, and 11RS each showed a band corresponding to a molecular weight of approximately 33,32,30,30, and 35 kDa, respectively (FIG. 12), higher than the theoretical molecular weights (monoisotopic mass) of 26,068.1, 26,584.4, 25,565.9, 26,082.1, 31,669.86 Da, respectively. Typically, SDS-PAGE gels, although useful to assess purity, may not characterize the true size of silk-based polymers due to the hydrophobic nature of the protein. Prince et al., 1995. The results of MALDI-TOF, however, showed 26,068.1, 26,584.4, 25,565.9, 26,082.1, 31,669.86 Da, respectively, confirming that the bioengineered proteins were the expected recombinant silk proteins. The recombinant proteins showed the theoretical pI of 10.6 and were soluble in water (5.0 mg/mL) at room temperature.

Characterization of pDNA complexes: DNA-Protein complex formation with DNA encoding luciferase with the five types of recombinant silk proteins (RS, RSR, SR, S2R, and 11RS) was characterized by AFM, DLS, and zeta-potential meter.

The hydrodynamic diameters of DNA complex of the recombinant silk-polylysine-RGD were measured by DLS (Table 4).

TABLE 4
Average diameters (nm) and distribution (PDI) of pDNA
complexes of the recombinant silk-RGD determined by DLS.

| N/P^{a} | RS | RSR | SR | S2R | 11RS |
|---|---|---|---|---|---|
| 0.1 | 2030 | 1230 | 2650 | 2490 | 2670 |
| | (0.456) | (0.275) | (0.675) | (0.352) | (0.362) |
| 1 | 676 | 567 | 948 | 498 | 693 |
| | (0.360) | (0.260) | (0.601) | (0.879) | (0.564) |
| 2 | 382 | 273 | 565 | 207 | 186 |
| | (0.475) | (0.387) | (0.533) | (0.357) | (0.415) |
| 5 | 360 | 305 | 419 | 181 | 190 |
| | (0.132) | (0.385) | (0.672) | (0.523) | (0.659) |
| 10 | 428, 2070 | 226, 681 | 400, 1530 | 201 | 172, 861 |
| | (—)^{b} | (—)^{b} | (—)^{b} | (0.814) | (—)^{b} |
| Polymer | 464 | 380 | 437 | 284 | 162 |
| | (0.692) | (0.600) | (0.658) | (0.453) | (0.416) |

^{a}N/P ratio refers to the ratio of number of amines to phosphate of pDNA
^{b}PDIs were not determined precisely because of their bimodal

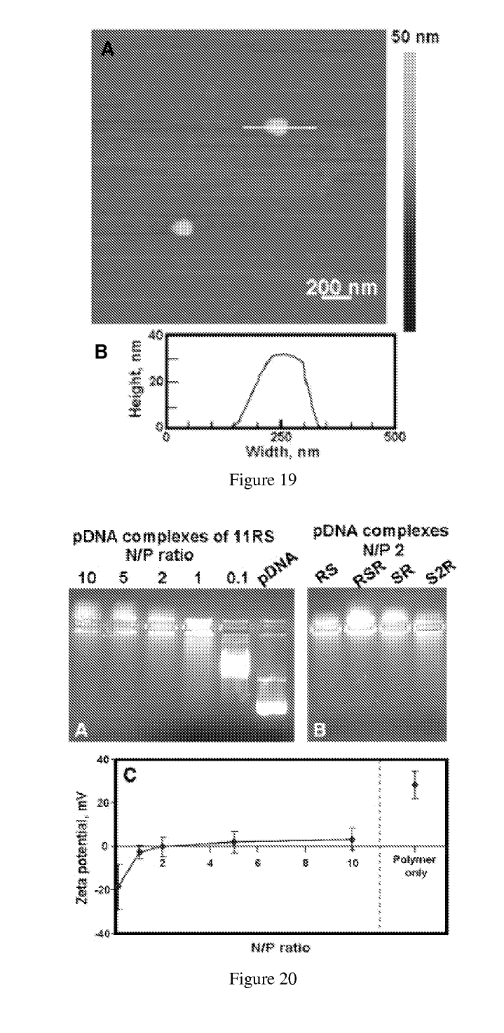

The average diameters of the complexes decreased with an increase in N/P ratio. The pDNA complexes prepared at a high N/P ratio, such as N/P 10, however, demonstrated bimodal distributions of their diameters. The pDNA complexes of the recombinant silk-RGD prepared at N/P 2 were cast on mica and observed by AFM (FIG. 19). All the complexes formed globular complexes, as shown in FIG. 19A. The pDNA complexes of 11 RS at N/P 2 demonstrated an average diameter and height of 223±32 nm and 30±8 nm, respectively (n=30), determined by AFM observations. The dimensions determined by DLS and AFM showed reasonable agreements considering the volume of the pDNA complex particles.

Agarose gel electrophoresis experiments were performed to investigate the interaction properties and electrolytic stabilities of the complexes of DNA and recombinant silk-polylysine-RGD. FIG. 20A shows the migration of free DNA and the DNA complexes of 11RS with various N/P ratios ranging from 0.1 to 10 in 1% agarose gels. The DNA complexes with 11RS at N/P 0.1 and 1 migrated to the same direction as free pDNA, whereas the DNA complexes at N/P over 2 migrated in the opposite direction or did not migrate from the well. These results indicate that the DNA complexes with 11RS at N/P below 1 were negatively charged, while the DNA complexes at N/P over 2 were positively charged. The DNA complexes of the other four recombinant silk-polylysine-RGD (RS, RSR, SR, S2R) prepared at N/P 2 were also characterized by agarose gel electrophoresis, and all samples demonstrated positive charges (FIG. 20B). To measure the values of the positive charge, the zeta potential of the pDNA complexes was determined. FIG. 20C shows the zeta potential of DNA complexes of 11RS with varying N/P ratios. The zeta potential increased with N/P ratio, and became positive at the N/P of 2. The zeta potential of the pDNA complexes of 11RS prepared at N/P 2 was 0.1±4.5 mV.

Cytotoxicity of DNA complexes with each of RS, RSR, SR, S2R, and 11RS at the N/P ratio of more than 10 was measured using the MTT assay. FIG. 20 shows that the complexes of all samples exhibited no cytotoxicity to HEK cells at higher concentration than that used in the transfection experiments (1.9 mg/mL).

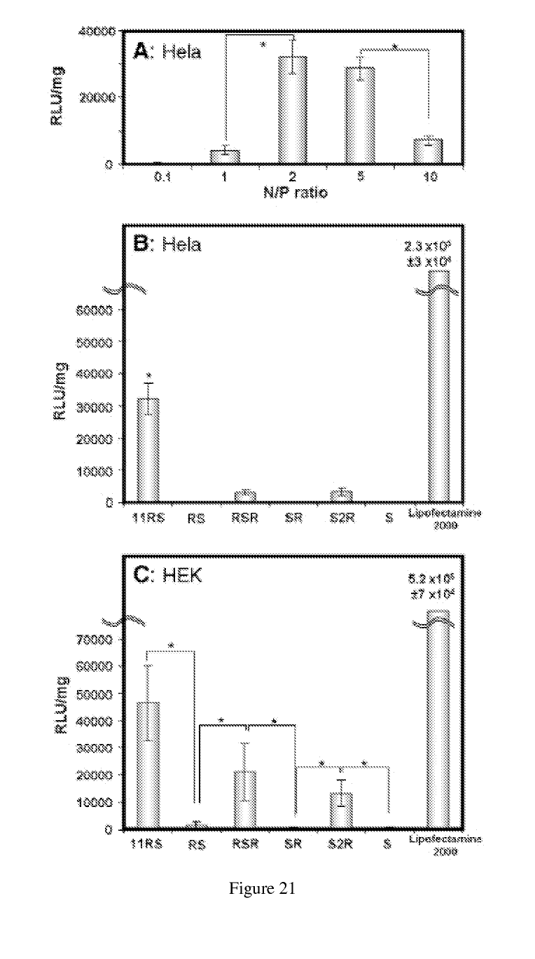

DNA transfection to HeLa and HFK cells: In vitro transfection experiments were performed with HeLa and HEK cells in order to evaluate the feasibility of the pDNA complexes with the cationic recombinant silks containing RGD peptides for nucleic acid delivery via integrin-mediated endocytosis. For a comparison of pDNA transfection efficiency of DNA complexed with various recombinant silk (11RS, RS, RSR, SR and S2R) at different N/P ratios, HeLa cells were transfected with luciferase pDNA as a reporter gene. FIG. 21A shows the transfection efficiencies to HeLa cells for pDNA complexes of 11RS with N/P ratios ranging from 0.1 to 10 based on the luciferase assays (n=4). pDNA complexes of 11RS prepared at N/P 2 demonstrated the highest transfection efficiency among the different N/P ratios, followed by a steep decrease in efficacy, perhaps due to excess recombinant silk interacting with the cells. FIGS. 21B and 21C show the transfection efficiencies to HeLa and HEK cells for pDNA complexed with various recombinant silk (11RS, RS, RSR, SR and S2R) at N/P of 2 as well as Silk6mer-30lys block copolymer (Sin the figures) and Lipofectamine 2000 as controls. Compared with recombinant silk containing RGD sequences, silk6mer-30lys block copolymers, which contained no RGD sequence, did not show substantial transfection to HeLa and HEK cells. The relative order of the transfection efficiency at N/P 2 decreased as follows: 11RS>RSR≈S2R>RS≈SR. Additionally, the pDNA complexes of 11RS exhibited significantly higher transfection efficiency to HeLa cells in comparison to the pDNA complexes of other recombinant silk contain one or two RGD sequences at N/P 2 (FIG. 21B). On the other hand, the pDNA complexes of 11RS did not show significantly higher transfection efficiency to HEK cells in comparison to RSR and SR2 (FIG. 21C).

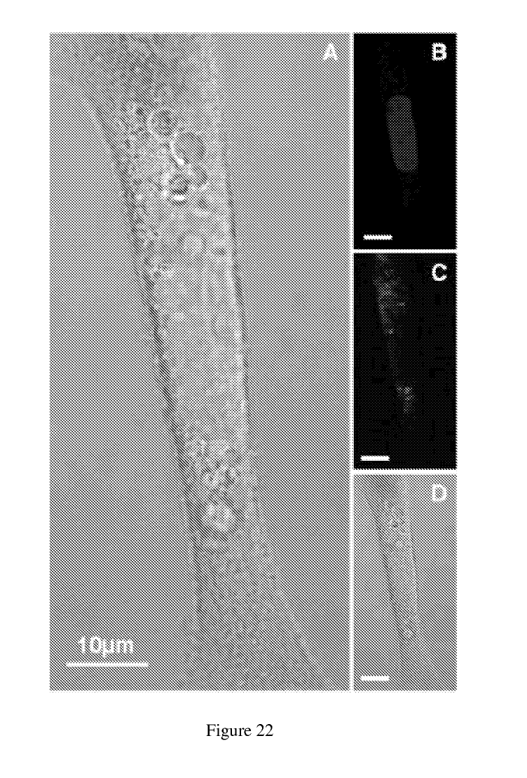

The intracellular distribution of the complexes of 11RS with Cy5- labeled pDNA and the nuclei stained with DAPI were investigated by CLSM. FIG. 22 shows typical CLSM images of HeLa cells incubated with the pDNA complexes. The Cy5-labeled pDNA (red) was distributed near the cell membrane as well as around the nuclei (blue), indicating that the pDNA was transferred near the nucleus via the 11 RS recombinant proteins.

Nucleic acid complexes of recombinant silk molecules containing cell-binding motifs for pDNA nucleic acid delivery were designed. How location and content of the cell-binding domain impacted the nucleic acid delivery of the complex was investigated. Five types of recombinant silks, RS, RSR, SR, S2R, and 11RS were cloned, expressed, and purified from E. coli. Globular nano-sized ion complexes of silk molecules containing 30 lysines were prepared and then complexed with pDNA (FIG. 19). pDNA complexes were incubated for 24 hr before the characterization of pDNA complexes and transfection experiments to obtain homogeneous pDNA complexes in size, since pDNA complexes right after the preparation showed bimodal size-distribution and almost no transfection efficiency. The average diameters of pDNA complexes of RS, RSR, SR, S2R, and 11RS at N/P 2 were 382, 315, 565, 207 and 186 nm, respectively, according to DLS measurements (Table 4). The average diameter of the pDNA complexes decreased with an increase in the N/P ratio (Table 4), suggesting the sizes of the complexes can be controlled by the N/P ratio. Also, no pDNA was released from the complexes during electrophoresis (FIGS. 20A and 20B), indicating that pDNA was packed inside the globular complexes.

The transfection experiments into HeLa and HEK cells with the DNA complexed with various recombinant silks at different N/P ratios revealed that the pDNA complex of 11RS prepared at N/P of 2, which was slightly positively charged (0.1±4.5 mV) and 186 nm in diameter, was more efficient than other complexes of the recombinant silks prepared herein (FIG. 21). The pDNA complexes of 11RS prepared at N/P of 10 showed lower transfection efficiency in comparison with N/P of 5 and N/P of 2, perhaps due to their bimodal size distribution, as listed in Table 4. DNA complexes of S2R and RSR, which contained two RGD sequences at different locations of recombinant silk sequence, showed almost the same transfection efficiency to HeLa cells. DNA complexes of RS and SR, which contained only one RGD sequence, demonstrated slightly lower transfection efficiency in comparison to S2R and RSR. These results suggest that the position of RGD motif, at the N-terminus or C-terminus, did not appear to influence the transfection efficiency of the pDNA complexes with the recombinant silk block copolymers. In other words, the recombinant silk molecules in the pDNA complexes were considered to be randomly assembled with pDNA and RGD sequences on the surface of the complexes, as shown in FIG. 10. Without being bound by theory, other functional peptides can also be added into different positions of the sequences of the delivery systems in order to construct additional novel protein vectors.

The RGD sequence has been used as a ligand to enhance cell-binding and cell transfection efficiency of nucleic acid vectors, because of selective recognition and binding αvβ3 and αvβ5 integrins, which have been reported to be expressed in HeLa cells. Oba et al., 2007; Kim et al., 2005; Connelly et al., 2007; Renigunta et al., 2006; Sun et al., 2008; Moore et al., 2008; Ishikawa et al., 2008; Quinn et al., 2009; Singh et al., 2003. Without being bound by theory, the transfection efficiency of pDNA complexes may also depend on the type of cells. In the case of HEK cells, the transfection may happen more easily than HeLa cells, independent of the type of DNA complexes (e.g., LIPOFECTAMINE™ 2000 transfection reagent versus poly(ethylene glycol)-polylysine block copolymer, as reported previously. Oba et al., 2007). Hence, the transfection efficiency of different nucleic acid vectors should be compared in the same cell line to better determine the effect of RGD sequences.

The addition of 11 RGD sequences into the recombinant silk (11RS) significantly enhanced the transfection efficiency of DNA into HeLa cells, whereas the other recombinant silk containing less RGD sequences did not significantly enhance transfection efficiency into HeLa cells (FIG. 21B). On the other hand, the recombinant silk with 11 RGD sequences (11RS) did not significantly enhance the transfection efficiency to HEK cells in comparison to the recombinant silks with two RGD sequences (RSR and SR2) (FIG. 21C). Therefore, the 11 RGD sequences in the recombinant silk appeared to induce RGD-integrin mediated transfection of DNA complexed therewith, though the other RGD sequences, dimeric and monomeric RGD sequences, did not induce RGD-integrin mediated transfection. Moreover, CLSM observation of the HeLa cells incubated with the Cy5-labeled pDNA complexes indicated pDNA was delivered to near the nucleus with the 11RS recombinant silk proteins (FIG. 22).

In summary, these results indicated that pDNA can be transferred to the nucleus with the recombinant silk proteins, 11RS, via the integrin-mediated endocytosis. The 11RGD sequences seemed to be sufficient for the integrin-mediated transfection. In consideration of the strength of ionic interactions between pDNA and polylysine sequences as shown in FIG. 20, the ionic interactions seemed strong enough to maintain the pDNA complexes even when DNA complexes were located inside cells. The pDNA can therefore be released from the complexes after partial degradation of the recombinant silk proteins by lysosome proteolytic activity.

Complex of PEI and RGD peptides were investigated and showed higher transfection efficiency to HEK cells in comparison with PEI molecules alone. The cytotoxicity of the complexes of PEI and RGD peptides was approximately 50% at the concentration of 400 μg/mL. Sun et al., 2008. Poly(ethylene glycol) (PEG)-based vectors demonstrated almost no cytotoxicity to HEK cells, and also exhibited comparable transfection efficiency to PEI. Moore et al., 2008. The addition of RGD into the platform of chemical synthesis of delivery polymers has been found to require multi-reaction steps with yields around 57%. Oba et al., 2007. In the embodiments of this invention, the recombinant silks are synthesized using genetic techniques, a one-step synthesis with monodisperse polymer chains as a result.

The pDNA complexes from the recombinant silks showed no cytotoxicity to HEK cells at the highest concentrations used in the transfection experiments (1.9 mg/mL), while also exhibiting the integrin-mediated transfection by 11RGD sequences. Further, the recombinant silks can be designed to add any number of peptides in selected positions and numbers to the silk carrier molecules. In this respect, this recombinant silk-base nucleic acid delivery system offers both benefits and options for general polymer-based nucleic acid delivery systems. In order to further enhance the efficiency and specificity of nucleic acid delivery, the recombinant silks prepared herein can be further modified with multi functional peptides, such as for cell-penetration and tumor-homing peptides. In particular, one of the highest transfection efficiencies of pDNA complexes with cell-penetrating peptides was reported to be approximately 45-fold higher in comparison to the pDNA complex of PEI at low DNA concentration (125 ng/mL) and without the specific penetrating peptide. Rittner et al., 2002. The recombinant silks modified to contain polylysine charged complexes and cell targeting domains, such as RGD, thus are a new platform polymer, like PEG, for non-viral nucleic acid delivery, but with tremendous versatility in design and function.

### Example 14

### Silk-Based Nucleic acid Carriers with Cell Membrane-Destabilizing Peptides

Design and cloning of silk sequence containing polylysine and ppTG1: The spider silk repeat unit was selected based on the consensus repeat (SGRGGLGGQGAGAAAAAGGAGQGGYGGLGSQGT) derived from the native sequence of the dragline protein MaSp1 sequence from the spider Nephila clavipes (Accession P19837). The Silk6mer-30lys containing six contiguous copies of this repeat and 30 lysines was developed through the transfer of cloned inserts to pET-30a, according to procedures published previously. Numata et al., 30 Biomats. 5775-84 (2009); Ferrari et al., 8 Mol. Ther. 284-94 (2003); Rabotyagova et al., 10 Macromol. Biosci. 49-59 (2010). The sequences of the synthetic oligonucleotides encoding ppTG1 residues were as follows: ppTG1-a: 5′-CTAGCGGCCTGTTTAAAGCGCTGCTGAAACTGCTGAAAAGCCTGTGGAAACTG CTGCTGAAAGCGA-3′, ppTG1-b: 5′-GCCGGACAAATTTCGCGACGACTTTGACGACTTTTCGGACACCTTTGACGACG ACTTTCGCTGATC-3′. The restriction sites for SpeI are italicized. ppTG1-a and ppTG1-b are complementary oligonucleotides which were annealed to form double stranded DNA. The double stranded DNAs of ppTG1 sequences were ligated into pET30-Silk6mer-30lys to generate pET30-Silk6mer-30lys-ppTG1(s), as shown in FIG. 23B, by DNA ligase (New England Biolabs Inc, Ipswich, Mass.).

Expression and purification of silk protein including polylysine and ppTG1: The constructs pET30-Silk6mer-30lys-ppTG1 monomer and pET30-Silk6mer-30lys-ppTG1 dimer were used to transform E. coli strain RY-3041, and the expression and purification of these proteins were carried out by methods reported previously. Numata et al., 2009; Numata et al., 6 Macromol. Biosci. 41-50 (2006); Arai et al., 91 J. Appl. Polym. Sci. 2383-90 (2004). SDS-polyacrylamide gel electrophoresis (PAGE) was performed using 4%-12% precast NuPage Bis-Tris gels (Invitrogen, Carlsbad, Calif.). The gel was stained with Colloidal blue (Invitrogen, Carlsbad, Calif.). Purified samples were extensively dialyzed against Milli-Q water. For dialysis, Spectra/Por Biotech Cellulose Ester Dialysis Membranes with MWCO of 100-500 Da (Spectrum Laboratories Inc, Rancho Dominguez, Calif.) were used. The recombinant proteins were further characterized to confirm sequence and molecular weight at the Tufts University Core Facility by Matrix Assisted Laser Desorption/Ionization-Time of Flight (MALDI-TOF) mass spectrometry.

Preparation and characterization of the DNA complexes of the recombinant silk containing polylysine and ppTG1: Two types of pDNAs encoding Green Fluorescence Protein, (GFP, 7650 bp) or Firefly Luciferase (Luc, 7041 bp) were amplified in competent DH5α E. coli (Invitrogen) and purified using EndoFree Plasmid Maxi Kits (Qiagen, Hilden, Germany). The DNA concentration was determined by absorbance at 260 nm. To prepare the complexes of the recombinant silk proteins with pDNA, a solution containing the silk protein (0.1 mg/mL) was mixed with the pDNA solution (370 μg/mL) at various N/P ratios. Here, N/P ratio refers to the ratio of number of amines to phosphates in pDNA. The mixture of recombinant silk and pDNA was incubated at room temperature (˜20° C.) overnight prior to characterization. To induce more beta-sheet structure, the pDNA complexes were collected by centrifugation, the supernatant was removed, and then methanol-treated pDNA complexes were obtained after incubation of the pDNA complexes in 50% methanol solution for 24 h. The pDNA complexes were characterized by zeta potential (Zetasizer Nano-ZS, Malvern Instruments Ltd, Worcestershire, UK), AFM (Dimension V, Veeco Instruments Inc, Plainview, N.Y.), and FTIR-ATR (JASCO FT/IR-6200) equipped with a multiple-reflection, horizontal M1Racle ATR attachment (using a Ge crystal, from Pike Tech, Madison Wis.). The pDNA complex solution (around 70 μL) was added to ultra pure water (450 μL, Invitrogen) and then used as a sample for zeta potential and size measurement. Zeta potential and zeta deviation of samples were measured three times by a zeta potential meter, and the average data were obtained using Dispersion Technology Software version 5.03 (Malvern Instruments Ltd). The pDNA complex solution was cast on cleaved mica, and observed in air at room temperature using a 200-250 μm long silicon cantilever with a spring constant of 2.8 N/m in tapping mode AFM. Calibration of the cantilever tip-convolution effect was carried out to obtain the true dimensions of objects by previously reported methods. Li et al., 24 Biomats. 357-65 (2003).

DNase resistance: The pDNA complexes were incubated with 100 μL of PBS containing 1 unit of DNase I (Sigma-Aldrich, St. Louis, Mo.) at 37° C. for 1 h. The digestion reactions were stopped by addition of 20 μL of 0.5 M EDTA at 20° C. The pDNA complexes were also treated with protease XIV or alpha-chymotrypsin (150 μg/mL) at 37° C. for 2 h. For agarose gel electrophoresis of the degradation products, 20 μL of each sample was mixed with loading buffer and analyzed on 1% agarose gel containing ethidium bromide (TAE buffer, 100V, 60 min).

Cell culture, transfection, and viability: Human embryonic kidney (HEK) cells (293 FT), which have been extensively used as an expression tool for recombinant proteins, were used as a model cell line. Ross & Hui, 6 Gene Ther. 651-59 (1999). The MDA-MB-435 melanoma cell line was also used to compare with HEK cells. Cultures were grown to confluence using media consisting of Dulbecco's Modified Eagle Medium (DMEM), 10% FBS, 5% glutamine, 5% Non-Essential Amino Acid (NEAA). The cultures were detached from their substrates using 0.25% trypsin (Invitrogen), and then replated in the 24-multiwell plate at a density of 70,000 cells/well. pDNA (1.2 μg) and recombinant silk (appropriate amount) complexes were added into each well. After incubation of the cells for 6 h at 37 ° C., the media was exchanged to the media without pDNA complexes. After another incubation for 72 h, fluorescence images were obtained by fluorescence microscopy (Leica Microsystems, Wetzlar, Germany) to evaluate GFP plasmid transfections. To evaluate luciferase gene expression quantitatively, a Luciferase assay (Promega, Madison, Wis.) was performed (n=4). The amount of protein in each well was determined using a BCA protein assay (Pierce Biotechnology, Rockford, Ill.), and then the relative light units (output)/weight of protein (RLU/mg) was obtained. Lipofectamine 2000 (Invitrogen) was used as a positive control vector. For cell viability, HEK cells (30,000 cells/well) were seeded into the 96-wells plates containing the pDNA complexes and cultured for 48 h in the media (100 μL) used in the transfection experiment. Cytotoxicity to HEK cells of the pDNA complexes was characterized by standard 3-(4,5-dimethylthiazol-2-yl)-5-(3-carboxymethoxyphenyl)-2-(4-sulfophenyl)-2H-tetrazolium (MTS) assay (Promega, Madison, Wis.) according to the manufacturer's instructions (n=8).

Statistical analysis: The particle sizes on mica substrates were measured by AFM using a Research Nanoscope software version 7.30 (Veeco Instruments Inc). The average value of 30 measurements was used. Statistical differences in particle sizes by AFM, cell transfection efficiency, and cell viability were determined by unpaired t-test with a two-tailed distribution and differences were considered statistically significant at p<0.05. The data in the AFM, cell transfection efficiency, and cell viability experiments are expressed as means±standard deviation.

Preparation of recombinant silk proteins: The recombinant silk proteins containing polylysine and ppTG1 sequences were expressed in Escherichia coli and purified with Ni-NTA chromatography. The domain structure and amino acid sequence of the spider silk variants generated with polylysine and the cell membrane destabilizing peptide (ppTG1) are shown in FIGS. 23A and 23B. Yields of the recombinant silk proteins were approximately 0.7 mg/L after purification and dialysis. The proteins after purification by Ni-NTA chromatography and dialysis were analyzed by SDS-PAGE and stained with Colloidal blue to evaluate purity. Silk-polylysine-ppTG1 monomer and dimer showed a major band corresponding to a molecular weight of approximately 33 and 34 kDa, respectively (FIG. 23C), higher than the theoretical molecular weights (monoisotopic mass) of 27,602.29 and 30,067.87 Da, respectively. Typically, SDS-PAGE gels, although useful to assess purity, may not characterize the true size of silk-based polymer due to the hydrophobic nature of the protein. Numata et al., 30 Biomats. 5775-84 (2009); Prince et al., 34 Biochem. 10879-85 (1995). The results from MALDI-TOF mass spectrometry, however, showed 27,602.29 and 30,067.87 Da, respectively confirming that the bioengineered proteins were the expected recombinant proteins. The Silk-polylysine-ppTG1 monomer and dimer showed the theoretical pIs of 10.70 and 10.75, respectively, and were soluble in water (approximately 2.0 mg/mL) at room temperature.

Characterization of pDNA complexes: Ionic complexes formation with pDNA encoding luciferase and the recombinant silk proteins, Silk-polylysine-ppTG1 monomer and dimer, were characterized at different N/P ratios (the ratio of number of amines to phosphates in pDNA) by AFM, DLS, and zeta-potentialmeter.

The hydrodynamic diameter and zeta potential of the pDNA complexes of the recombinant silks were measured by zeta-nanosizer (Table 5).

TABLE 5
Average diameters, their distribution (PDI) and zeta potentials
of the pDNA complexes of the recombinant silk proteins

| | Average diameters [nm] | | | | Zeta potential [mV] | |
|---|---|---|---|---|---|---|
| | and PDI | | | | SL- | |
| Samples | SL-Monomer^{a} | | SL-Dimer^{a} | | Monomer^{a} | SL-Dimer^{a} |
| N/P 0.1 | 563 | (0.679) | 265 | (0.856) | −43.1 ± 3.9 | −33.4 ± 5.5 |
| N/P 1 | 326 | (0.862) | 187 | (0.720) | −37.9 ± 6.4 | −29.8 ± 5.5 |
| N/P 2 | 108 | (0.564) | 99 | (0.524) | −37.5 ± 7.1 | −26.2 ± 6.3 |
| N/P 5 | 48.7, 698 | (—)^{b} | 31.5, 575 | (—)^{b} | −36.9 ± 5.4 | −22.7 ± 6.2 |
| Silk proteins | 169 | (0.453) | 163 | (0.821) | −30.5 ± 6.9 | −13.9 ± 6.3 |
| without pDNA | | | | | | |
| MeOH-treated | 146 | (0.824) | 130 | (0.802) | −37.5 ± 5.1 | −29.6 ± 5.7 |
| (N/P 2) | | | | | | |

^{a}SL-Monomer: Silk-polylysine-ppTG1 monomer, SL-Dimer: Silk-polylysine-ppTG1 dimer.
^{b}PDIs were not determined precisely because of their bimodal distribution.

The average diameters of the Silk-polylysine-ppTG1 monomer and dimer at the concentration of 0.1 mg/mL without pDNA were 169 nm and 163 nm, respectively. The average diameters of the complexes decreased with an increase in N/P ratio, and the pDNA complexes prepared at an N/P of 5 demonstrated a bimodal distribution of their diameters. The zeta potential of pDNA complexes increased slightly with an increase in N/P ratio. The pDNA complexes of the Silk-polylysine-ppTG1 monomer showed a lower zeta potential in comparison to the Silk-polylysine-ppTG1 dimer, because of lower zeta potential of Silk-polylysine-ppTG1 monomer. Based on the average diameters and zeta potentials, the pDNA complexes with Silk-polylysine-ppTG1 monomer and dimmer prepared at an N/P of 2 are more suitable for in-vitro transfection. The average diameters and zeta potentials for the pDNA complexes with Silk-polylysine-ppTG1 monomer and dimmer prepared at an N/P of 2 were 108 nm and 99 nm, and −37.5±7.1 mV and −26.2±6.3 mV, respectively. Methanol treatment for 24 h, for inducing a protein transition to beta sheet for the silk domains, was performed on the pDNA complexes of the recombinant silk proteins containing both ppTG1 monomer and dimer (N/P 2). The results demonstrated slightly increased dimensions and PDIs for both complexes after methanol treatment, but the zeta potentials remained the same after methanol treatment (Table 5). The pDNA complexes of Silk-polylysine-ppTG1 dimer before and after the methanol treatment were also characterized by FTIR-ATR. As shown in FIG. 24, a peak at 1625 cm⁻¹ in the amide I region are present after the methanol treatment, indicating the beta-sheet structures (crystallization) in the recombinant silk protein in the pDNA complexes. Almofti et al., 20 Mol. Membr. Biol. 35-43 (2003).

The DNA complexes of Silk-polylysine-ppTG1 dimer prepared at an N/P of 2 were cast on mica and characterized by AFM. As shown in FIG. 25, the DNA complexes of Silk-polylysine-ppTG1 dimer prepared at an N/P of 2 formed homogeneous globular complexes. Based on the AFM observations, this DNA complexes of recombinant silk containing ppTG1 dimer at N/P 2 demonstrated an average diameter and height of 185±43 nm and 3.6±1.1 nm, respectively (n=30). The dimensions determined by DLS and AFM showed reasonable agreements considering the volume of the DNA complex particles,

Cytotoxicity of the pDNA complexes with an N/P ratio of 2 for Silk-polylysine-ppTG1 monomer and dimer was determined using the standard MTS assay. The pDNA complexes of Silk-polylysine-ppTG1 monomer and dimer at a concentration of approximately 100 μg/mL showed 75±3% and 69±8% of cell viability, respectively.

DNase resistance and nucleic acid release behavior: The stability of pDNA incorporated with the recombinant silk protein, Silk-polylysine-ppTG1 dimer, against DNase was characterized using DNase I treatment and agarose gel electrophoresis, as shown in FIG. 26. The results for all samples were compared with the result for the sample containing free pDNA only (FIG. 26, lane 1). For free pDNA, DNase I enzymatic treatment for 1 h degraded free pDNA rapidly (FIG. 26, lane 2), while no degradation was evident by protease XIV and alpha-chymotrypsin for 2 h (FIG. 26, lanes 3 and 4). For pDNA complexes of silk-polylysine-ppTGI dimer, there were still pDNA in the well after the DNase I treatment (FIG. 26, lane 5); and subsequently to the DNase I treatment, the pDNA in the silk-polylysine-ppTGI dimer was released from the complex after enzymatic treatment by protease XIV (FIG. 26, lane 6). Still for pDNA complexes of silk-polylysine-ppTGI dimer, a-chymotrypsin and protease XIV, hydrolases that digest silk proteins (Numata et al., 31 Biomats. 2926-33 (2010); Almofti et al., 20 Mol. Membr. Biol. 35-43 (2003); Bowman et al., 85 P.N.A.S. 7972-76 (1988)), released pDNA from the complexes (FIG. 26, lanes 7 and 8). The methanol-treated pDNA complexes also protected the incorporated pDNA from DNase I treatment for 1 h (FIG. 26, lane 9). The methanol-treated pDNA complexes after enzymatic treatment by alpha-chymotrypsin released less pDNA when compared with the treatment by protease XIV (FIG. 26, lanes 11 and 12), perhaps because the crystallized silk is less susceptible to this protease than the noncrystallized (non beta sheet) containing protein. Numata et al., 2010; Bowman et al., 1988; Huang et al., 278 J. Biol. Chem. 46117-23 (2003).

Nucleic acid transfection to cells: In vitro transfection experiments were performed with HEK cells and MDA-MB-435 cells to evaluate the feasibility of the pDNA complexed with the cationic recombinant silks containging the ppTG1 cell membrane destabilizing peptides for nucleic acid delivery. To determine the most efficient N/P ratio of the pDNA complexes, HEK cells were transfected via the Silk-polylysine-ppTG1 dimer with luciferase pDNA as a reporter gene. FIG. 27A shows the transfection efficiencies to HEK cells for pDNA complexes of Silk-polylysine-ppTG1 dimer with N/P ratios ranging from 0.1 to 5 based on the luciferase assays (n=4). pDNA complexes of Silk-polylysine-ppTG1 dimer prepared at N/P 2 demonstrated the highest transfection efficiency among the different N/P ratios, followed by a steep decrease in efficacy, perhaps due to excess recombinant silk interacting with the cells and pDNA. FIG. 27B shows the transfection efficiencies to HEK cells and MDA-MB-435 cells for pDNA complexes of the recombinant silk proteins containing both ppTG1 monomer and dimer(N/P 2), in comparison with the transfection reagent Lipofectamine 2000, as a control. The pDNA complexes of Silk-polylysine-ppTG1 dimer exhibited the same transfection efficiency to HEK cells as Lipofectamine 2000 and also showed significantly higher transfection efficiency to both cells in comparison to the Silk-polylysine-ppTG1 monomer at N/P 2. The transfection experiments with a GFP reporter gene to HEK cells and MDA-MB-435 cells were also performed, as shown in FIGS. 27C and 27D), indicating that the transfection of pDNA complexes and their degradation products inside the cells did not significantly influence cell morphology.

In vitro transfection behaviors in a course of 144 h using the pDNA complexes of Silk-polylysine-ppTG1 dimer (N/P 2) before and after methanol treatment were also characterized by luciferase assay, as shown in FIG. 28. Although the transfection efficiency was lower for the methanol-treated Silk-polylysine-ppTG1 dimer than the same complexes without methanol treatment, the methanol-treated pDNA complexes demonstrated slow and constant release of pDNA for at least 144 h (6 days).

1. A biomaterial nucleic acid complex comprising:
a recombinant silk protein repeating unit comprising a plurality of amino acids with positively charged R groups; and
a nucleic acid complexed with the recombinant silk protein via ionic interaction.

2-3. (canceled)

4. The biomaterial nucleic acid complex of claim 1, wherein the biomaterial nucleic acid complex is in a globular form.

5. The biomaterial nucleic acid complex of claim 1, wherein the recombinant silk protein of the biomaterial nucleic acid complex at least partially self-assembles to beta-sheet structure.

6-10. (canceled)

11. The biomaterial nucleic acid complex of claim 1, wherein the recombinant silk protein further comprises one or more functional peptide domains selected from the group consisting of signal peptides of virus, tumor-homing peptides, metal binding domain, cell targeting peptides, drug binding peptides, functional domains to alter cell activities, cell binding motifs, cell-penetrating and/or cell membrane-destabilizing peptides (CPPs), and combinations thereof.

12. The biomaterial nucleic acid complex of claim 1, wherein the nucleic acid is selected from the group consisting of DNA, cDNA, DNA vectors or plasmids, RNA vectors or plasmids, dsRNA, siRNA, shRNA, saRNA, mRNA, miRNA, pre-miRNA, ribozyme, antisense RNA, and combinations thereof.

13. A silk-based nucleic acid delivery system comprising a recombinant silk-based block copolymer complexed with a nucleic acid, wherein the copolymer comprises a repeating unit of a silk consensus sequence and a poly(L-lysine) domain.

14. The silk-based nucleic acid delivery system of claim 13, wherein the nucleic acid is selected from the group consisting of DNA, cDNA, DNA vectors or plasmids, RNA vectors or plasmids, dsRNA, siRNA, shRNA, saRNA, mRNA, miRNA, pre-miRNA, ribozyme, antisense RNA, and combinations thereof.

15. The silk-based nucleic acid delivery system of claim 13, wherein the silk consensus sequence contain the sequence of SGRGGLGGQGAGAAAAAGGAGQGGYGGLGSQGT (SEQ ID NO: 1) and the poly(L-lysine) domain contains one or more of 15 lys, and wherein the nucleic acid is a DNA.

16. The silk-based nucleic acid delivery system of claim 15, wherein the molar ratio of the recombinant silk protein to nucleotides of DNA (P/N) ranges from about 2.5 to about 50.

17. The silk-based nucleic acid delivery system of claim 16, wherein the recombinant silk-based block copolymer further comprises one or more functional peptide domains selected from the group consisting of cell penetrating peptides and/or cell membrane-destabilizing peptides (CPPs), signal peptides of virus, tumor-homing peptides, metal binding domain, cell targeting peptides, cell binding motifs, drug binding peptides, functional domains to alter cell activities, and combinations thereof.

18. The silk-based nucleic acid delivery system of claim 13, wherein the recombinant silk-based block copolymer is a 6mer of the silk consensus residues and a poly(L-lysine) domain of 30 lys, and wherein P/N is 10.

19. The silk-based nucleic acid delivery system of claim 13, wherein the recombinant silk-based block copolymer further comprises one or more RGD domains or one or more ppTG1 domains.

20. The silk-based nucleic acid delivery system of claim 19, wherein the ratio of numbers of amines to phosphates of DNA (N/P) ranges from about 2 to about 10.

21. The silk-based nucleic acid delivery system of claim 20, wherein the recombinant silk-based block copolymer is a 6mer of the silk consensus residues, a poly(L-lysine) domain of 30 lys, and a RGD domain of 11 RGD, and wherein N/P is 2.

22-23. (canceled)

24. The silk-based nucleic acid delivery system of claim 19, wherein the recombinant silk-based block copolymer is a 6mer of the silk consensus residues, a poly(L-lysine) domain of 30 lys, and a dimeric ppTG1, and wherein N/P is 2.

25. The silk-based nucleic acid delivery system of claim 13, wherein the complex is in a globular form with an average size ranging from about 50 nm to about 400 nm in diameter.

26-27. (canceled)

28. The silk-based nucleic acid delivery system of claim 13, wherein the complex is neutral or positively charged.

29-31. (canceled)

32. A method of transfecting a cell comprising contacting the cell with a silk-based nucleic acid delivery system of claim 13.

33-79. (canceled)

80. The biomaterial nucleic acid complex of claim 11, wherein the cell binding motif comprises one or more RGD residues.

81. The biomaterial nucleic acid complex of claim 11, wherein the cell-penetrating and/or cell membrane-destabilizing peptides (CPPs) comprises one or more ppTG1 sequences.


SEQUENCE LISTING (19 sequences)

SEQ ID NO: 1 - protein, 33 aa, Nephila clavipes
SGRGGLGGQGAGAAAAAGGAGQGGYGGLGSQGT

SEQ ID NO: 2 - DNA, 51 bp, Artificial Sequence
  Description of Artificial Sequence Synthetic oligonucleotide
ctagcaagaaaaagaaaaaaaagaaaaaaaagaaaaagaaaaaaaagaaaa

SEQ ID NO: 3 - DNA, 51 bp, Artificial Sequence
  Description of Artificial Sequence Synthetic oligonucleotide
ctagttttcttttttttctttttcttttttttcttttttttctttttcttg

SEQ ID NO: 4 - DNA, 15 bp, Artificial Sequence
  Description of Artificial Sequence Synthetic oligonucleotide
ctagccgaggcgaca

SEQ ID NO: 5 - DNA, 15 bp, Artificial Sequence
  Description of Artificial Sequence Synthetic oligonucleotide
ctagtgtcgcctcgg

SEQ ID NO: 6 - DNA, 66 bp, Artificial Sequence
  Description of Artificial Sequence Synthetic oligonucleotide
ctagcggcctgtttaaagcgctgctgaaactgctgaaaagcctgtggaaactgctgctgaaagcga

SEQ ID NO: 7 - DNA, 66 bp, Artificial Sequence
  Description of Artificial Sequence Synthetic oligonucleotide
gccggacaaatttcgcgacgactttgacgacttttcggacacctttgacgacgactttcgctgatc

SEQ ID NO: 8 - protein, 246 aa, Artificial Sequence
  Description of Artificial Sequence Synthetic polypeptide
MHHHHHHSSGLVPRGSGMKETAAAKFERQHMDSPDLGTDDDDKAMAASGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTS

SEQ ID NO: 9 - protein, 17 aa, Artificial Sequence
  Description of Artificial Sequence Synthetic peptide
KKKKKKKKKKKKKKKTS

SEQ ID NO: 10 - protein, 34 aa, Artificial Sequence
  Description of Artificial Sequence Synthetic polypeptide
KKKKKKKKKKKKKKKTSKKKKKKKKKKKKKKKTS

SEQ ID NO: 11 - protein, 51 aa, Artificial Sequence
  Description of Artificial Sequence Synthetic polypeptide
KKKKKKKKKKKKKKKTSKKKKKKKKKKKKKKKTSKKKKKKKKKKKKKKKTS

SEQ ID NO: 12 - protein, 53 aa, Artificial Sequence
  Description of Artificial Sequence Synthetic polypeptide
MHHHHHHSSGLVPRGSGMKETAAAKFERQHMDSPDLGTDDDDKAMAASVASAS

SEQ ID NO: 13 - protein, 233 aa, Artificial Sequence
  Description of Artificial Sequence Synthetic polypeptide
SGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSKKKKKKKKKKKKKKKTSKKKKKKKKKKKKKKKTS

SEQ ID NO: 14 - protein, 290 aa, Artificial Sequence
  Description of Artificial Sequence Synthetic polypeptide
MHHHHHHSSGLVPRGSGMKETAAAKFERQHMDSPDLGTDDDDKAMAASVASASRGDTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSKKKKKKKKKKKKKKKTSKKKKKKKKKKKKKKKTS

SEQ ID NO: 15 - protein, 295 aa, Artificial Sequence
  Description of Artificial Sequence Synthetic polypeptide
MHHHHHHSSGLVPRGSGMKETAAAKFERQHMDSPDLGTDDDDKAMAASVASASRGDTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSKKKKKKKKKKKKKKKTSKKKKKKKKKKKKKKKTSRGDTS

SEQ ID NO: 16 - protein, 284 aa, Artificial Sequence
  Description of Artificial Sequence Synthetic polypeptide
MHHHHHHSSGLVPRGSGMKETAAAKFERQHMDSPDLGTDDDDKAMAAGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSKKKKKKKKKKKKKKKTSKKKKKKKKKKKKKKKTSRGDTS

SEQ ID NO: 17 - protein, 289 aa, Artificial Sequence
  Description of Artificial Sequence Synthetic polypeptide
MHHHHHHSSGLVPRGSGMKETAAAKFERQHMDSPDLGTDDDDKAMAAGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSKKKKKKKKKKKKKKKTSKKKKKKKKKKKKKKKTSRGDTSRGDTS

SEQ ID NO: 18 - protein, 345 aa, Artificial Sequence
  Description of Artificial Sequence Synthetic polypeptide
MHHHHHHSSGLVPRGSGMKETAAAKFERQHMDSPDLGTDDDDKAMAASVASASRGDTSRGDTSRGDTSRGDTSRGDTSRGDTSRGDTSRGDTSRGDTSRGDTSVASASRGDTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTXGRGGLGGQGAGAAAAAGGAGQGGYGGLGSQGTSKKKKKKKKKKKKKKKTSKKKKKKKKKKKKKKKTS

SEQ ID NO: 19 - protein, 324 aa, Artificial Sequence
  Description of Artificial Sequence Synthetic polypeptide
MHHHHHHSSGLVPRGSGMKETAAAKFERQHMDSPDLGTDDDDKAMAASGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSKKKKKKKKKKKKKKKTSKKKKKKKKKKKKKKKTSGLFKALLKLLKSLWKLLLKATSGLFKALLKLLKSLWKLLLKATS